(19) United States
(12) Patent Application Publication
(10) Pub. No.: US 2023/0151386 A1
(43) Pub. Date: May 18, 2023

(54) Apparatus for Improved Transfection Efficiency and / or Protein Expression and Method of Use Thereof

(71) Applicant: St Andrews Pharmaceutical Technology Ltd., Henley-On-thames, Oxfordshire (GB)

(72) Inventors: William J. Henry, St Andrews (GB); Anna Montali, Merano (BZ) (IT); Jean-Christophe Bourdon, Dundee (GB)

(21) Appl. No.: 17/912,928

(22) PCT Filed: Mar. 25, 2021
(86) PCT No.: PCT/GB2021/050737

§ 371 Date: Sep. 20, 2022

(30) Foreign Application Priority Data
Mar. 26, 2020 (GB) 2004411.1
Mar. 26, 2020 (GB) 2004412.9
Jun. 18, 2020 (GB) 2009296.1
Jun. 18, 2020 (GB) 2009297.9

(51) Int. Cl. C12N 15/85; C12M 1/42

(52) U.S. Cl. CPC C12N 15/85; C12M 35/02; C12N 2310/10; C12N 2529/00

(57) ABSTRACT

A method and apparatus for improving transfection efficiency in eukaryotic cells including the steps of providing a transfection mixture including an agent associated with at least one amphiphilic construct suitable for transfection. Adding the transfection mixture to one or more eukaryotic cells to form a transfection complex and allowing the transfection complex to undergo a transfection process to form one or more transfected cells. The method also includes the step of directing pulsed electromagnetic signals provided at any or any combination of a pre-determined frequency, at a pre-determined pulse rate, or at a pre-determined power, at the transfection mixture at step (a) prior to creating the transfection complex, at the transfection complex in step (b), at the transfection complex in step (c) and/or at the transfected cell complex after step (c).

The present invention relates to apparatus for achieving improved transfection efficiency and/or protein expression and a method of use thereof. The apparatus can also be used to improve protein expression in cells and a method of use thereof.

Although the following description makes reference to how apparatus for allowing improved transfection of cells can be used for the purposes of gene therapy, it will be appreciated by persons skilled in the art that the present invention can be used for any purpose or application where the transfection of cells is required, such as in the production of viral vectors, gene therapy or modification, protein expression, autologous cell therapy and/or the like. It will also be appreciated that the apparatus and methods of the present invention can be undertaken in respect of in vitro cells, ex vivo cells and/or in vivo cells.

Transfection is a process by which nucleic acid is introduced into eukaryotic cells. Transfection can be stable, in that the transfected nucleic acid may be continuously expressed and is passed on to daughter cells. Alternatively, transfection can be transient, in that the transfected nucleic acid is only expressed for a short period of time following the transfection and is not passed on to daughter cells. The use of either type of transfection in the field of gene therapy is well known [1] and focuses on the utilization of the therapeutic delivery of nucleic acid into a patients cells to act as a drug for the treatment of a disease. For example, the purpose can be to replace faulty genes in a patient that, if not treated, could lead to the patient suffering from gene related and inherited conditions. In the laboratory setting, immortal cell lines are often transfected with an exogenous gene, typically in the form of a plasmid. Following transfection, the successfully transfected cells will express the exogenous gene.

In a transient transfection, an exogenous gene (typically encapsulated in a carrier such as polyethylenimine (PEI)) will be introduced to a population of cells. A portion of these cells will be successfully transfected, and will begin to express the exogenous gene. After a short period of time, the level of expression will fall, and the cells are typically processed or otherwise discarded at this point.

In a stable transfection, the cells are transfected as above. A portion of the cells will have integrated the exogenous gene in a stable manner. The stably transfected cells can be isolated and selected from the population of cells based upon expression of the exogenous gene, and these cells propagated to produce an immortal cell line expressing the exogenous gene over a longer period of time.

Transfection efficiency (i.e. the rate at which cells are successfully transfected with an exogenous gene) is typically low in the prior art methods. Multiple strategies have been adopted to try to increase the transfection efficiency of cell lines (e.g. electroporation, specialised reagents for transfection, and others). What is needed is apparatus and a method for further improving transfection protocols in order to improve the transfection efficiency of any given transfection protocol.

A recent development has been to manipulate the genetic sequence of a patients own immune cells to transform them into cells that will recognise and attack specific cancerous cells within the patients body [2]. Approaches where a patients cells are genetically manipulated is known as ‘gene therapy’. Once promising avenue of gene therapy for treatment of cancer is the genetic manipulation of T-cells such that they express chimeric antigen receptors which allow the T-cells to target cancerous tissue growth more effectively. Such cells are known as chimeric antigen receptor T-cells (“CAR-T” cells) and the therapy is known as “CAR-T cell therapy”. The immune cells are first removed from the patients body and then undergo a transfection process ex vivo that converts the cells to cancer-seeking killer cells. The transfected cells are then re-administered to the patient to treat their cancer. The transfection of these cells is typically achieved using an approach involving associating the exogenous genetic material with a carrier molecule, such as a nanoparticle or a liposomal carrier.

An example of a conventional transfection process includes the step of encapsulating target DNA in a phospholipid, bilayer vesicle or liposome that is then administered into a eukaryotic cell [3]. As the liposome is formed of phospholipid, the liposome has an affinity for eukaryotic cell membranes that, likewise, have a phospholipid bilayer, and so there is fusion of these systems. External DNA can therefore be transferred via this fusogenic mechanism into the eukaryotic cell and become extrachromosomal genetic information for the cell. A simple conventional transfection process involves encapsulating the exogenous nucleic acid (e.g. DNA plasmid containing the gene of interest) in a cationic polymer (PEI) [6]. While there are potentially significant advantages of such processes, these conventional processes are slow and have a poor transfection efficiency. The low transfection efficiency of these methods makes then wasteful and time consuming, thus expensive.

It is therefore an aim of the present invention to provide apparatus that improves transfection efficiency and/or protein expression in eukaryotic cells that overcomes the abovementioned problems.

It is a further aim of the present invention to provide a method of improving transfection efficiency and/or protein expression in eukaryotic cells.

It is a further aim of the present invention to provide transfection enhancing apparatus and/or to a method of use thereof.

It is a yet further aim of the present invention to provide apparatus that improves the effectiveness of gene therapy and/or therapeutic treatment in animals or humans.

It is a yet further aim of the present invention to provide a method of improving the effectiveness of gene therapy and/or therapeutic treatment in animals or humans.

It is a yet further aim of the present invention to provide apparatus that improves the production of viral vectors and/or a method of use thereof.

It is a yet further aim of the present invention to provide apparatus that improves protein expression in human and/or animals cells and/or a method of use thereof.

A further aim of the present invention is to allow the speed of preparation and/or application of transfection material to be improved and a yet further aim is to allow an increased yield of transfected cells.

It is a yet further aim of the present invention to provide apparatus and/or method of use which allows for the delivery of an agent, drug and/or therapeutic treatment through the skin of a patient in a more targeted and efficient manner at lower cost.

It is a further aim of the present invention to provide apparatus and/or method of use which can be used to provide the delivery of an agent, drug and/or a therapeutic treatment to a patient which allows the apparatus to be easily portable and/or used in a patients home.

According to a first aspect of the present invention there is provided a method of improving transfection efficiency in eukaryotic cells, said method including the steps of:
a) providing a transfection mixture including an agent associated with at least one amphiphilic construct suitable for transfection;
b) introducing the transfection mixture to one or more eukaryotic cells to form a transfection complex;
c) allowing the transfection complex to undergo a transfection process to form one or more transfected cells;
characterised in that the method includes the step of directing pulsed electromagnetic signals provided at any or any combination of a pre-determined frequency, at a pre-determined pulse rate, and at a pre-determined power, at the transfection mixture at step a) prior to creating the transfection complex, at the transfection complex in step b), at the transfection complex in step c) and/or at the transfected cells complex after the transfection step c).

The Applicants have surprisingly found that the administration of pulsed electromagnetic (PEM) signals before, during and/or after transfection significantly increases the transfection efficiency and/or protein expression yield created by the transfection process. The transfection rate is significantly improved and allows for the enhanced frequency of transfected cells containing the agent and/or exogenous nucleic acid. Thus, the present invention provides a non-invasive, non-chemical approach to improving cell viability, gene transfer, transfection rate and/or protein production. The present invention enhances the transportation of extra-cellular material or agent from an environment external to a cell to an internal environment in the interior of the cell.

The term “pulsed electromagnetic signals” used herein is preferably defined as a sequence or pattern of signals in the electromagnetic spectrum range that change in amplitude from a base line to a higher or lower value, followed by a return to the base line or a return substantially to the base line. Further preferably the change in signal amplitude is rapid and transient and occurs in a repeating sequence. In one example, the base line represents an absence of electromagnetic signals being emitted from an electromagnetic signal source or transmission means. Preferably the base line is considered to be a rest or relaxation period for the cells and/or pulsed electromagnetic signals.

Preferably the method can take place entirely in-vitro, entirely in-vivo, or partially in-vitro and partially in-vivo. For example, the eukaryotic cells could be transfected in vitro and used for one or more purposes or applications in vitro. In a further example, the eukaryotic cells could be extracted from a patient, transfected in-vitro and then re-introduced back into the patient (this is interchangeably referred to as an “ex vivo” method. Alternatively, the transfection complex could be injected or otherwise transported into a patient and the patients cells could be transfected in-vivo.

Preferably the agent in the transfection mixture is any agent suitable for transfection and/or any or any combination of nucleic acid, a pharmaceutical and/or therapeutic agent or compound, an agent of therapeutic and/or pharmaceutical interest, a small molecule or small molecular material of less than 5 Kilodaltons, a large molecule or large molecular material greater than or equal to approximately 5 Kilodaltons, one or more proteins, vaccine, one or more antibodies, an organic agent and/or the like.

The term ‘pharmaceutical and/or therapeutic agent or compound’ preferably refers to compounds which are deployed or being developed for deployment into the clinic, which have a defined medicinal effect.

The term ‘agent of therapeutic and/or pharmaceutical interest’ preferably refers to compounds that have been developed for use and/or are being investigated for use in research and/or in the clinic. These agents or compounds may have a known mechanism of action, but the clinical suitability and relevance may not have been demonstrated or investigated. In some embodiments, the mechanism of action of these agents or compounds may not yet have been uncovered. Regardless, the underlying mechanism of the present invention allows superior intracellular delivery of these agents or compounds.

In one aspect, there is provided a method of intracellular delivery, said method comprising:
a) providing a first mixture including an agent associated with at least one amphiphilic construct suitable for intra-cellular delivery;
b) introducing the first mixture to one or more eukaryotic cells to form a second mixture comprising the first mixture and said one or more eukaryotic cells;
c) allowing the second mixture to undergo a process whereby the agent associated with at least one amphiphilic construct is delivered into said one or more eukaryotic cells;
characterised in that the method further comprises directing pulsed electromagnetic signals provided at any or any combination of a pre-determined frequency, at a pre-determined pulse rate, or at a pre-determined power:
   at the first mixture at a) prior to creating the second mixture;
   at the second mixture in b);
   at the second mixture in c); and/or
   at the one or more eukaryotic cells after step c).

In some embodiments, the agent is or comprises a nucleic acid, and transfection refers to the process by which the nucleic acid is introduced into the one or more eukaryotic cells, thus causing the cell(s) to express an exogenous nucleic acid (for example an exogenous RNA, DNA, RNA/DNA hybrid or a gene encoded by, or a protein expressed from, said nucleic acid). In other embodiments, where the agent does not comprise a nucleic acid, the word transfection is used interchangeably with the term ‘intracellular delivery’, and in this context means that the agent is delivered across the cell membrane into the cytoplasm of the one or more eukaryotic cells. In some embodiments, the method, particularly when the agent does not comprise a nucleic acid, can thus be considered to be a method of trans-membrane delivery.

In any aspect or embodiment described herein, unless otherwise apparent, the method may be considered to be a method of transfection, intracellular delivery or trans-membrane delivery.

In any aspect or embodiment described herein, unless otherwise apparent, the transfection mixture can be described as a first mixture, and/or the transfection complex can be described as a second mixture. The delivery to the cells may be considered to be trans-membrane, intracellular or intra-cytoplasmic delivery.

Preferably the agent is associated with the at least on amphiphilic construct in that it is contained within the amphiphilic construct, it forms a complex with the amphiphilic construct, it is contained on the amphiphilic construct, it is bonded to the amphiphilic construct and/or the like.

In one embodiment the eukaryotic cells could include any or any combination of adherence cells, suspension cells, blood cells, lymphocytes, granulocytes, T-cells and/or the like.

In some embodiments, the eukaryotic cells are suspended in solution, adhered to a substrate, or a mixture of both suspended and adhered cells.

In some embodiments, the eukaryotic cells are immortal cells or cells derived from an immortal cell line. For example, Chinese Hamster Ovary (CHO) cells, Human Embryonic Kidney (HEK) cells, Human Colon Tumour (HCT) 116 cells, or Jurkat E6 cells.

In some embodiments, the eukaryotic cells are the cells in or derived from the tissue of a human or animal subject. For example, cells may have been extracted from a subject to be transfected and then reintroduced to the subject. In some embodiments, the eukaryotic cells are derived from the blood of a subject. In some embodiments, the eukaryotic cells are T-cells, lymphocytes, granulocytes, macrophages and/or other white blood cells. In some embodiments, the T-cells are any or any combination of helper T-cells or cytotoxic T-cells. In some embodiments, the T-cells comprise CD4+ cytotoxic T lymphocytes and/or CD8+ cytotoxic T lymphocytes.

One exemplary use of the apparatus and method of the invention is adoptive T-cell therapy (ACT), involving the generation of so called ‘CAR-T’ cells. In such a technique, the apparatus and/or method are used on T-cells derived from a subject. The cells are cultured and transfected in vitro to express the chimeric antigen receptor, and then expanded in vitro prior to being reintroduced into the patient.

The present apparatus and/or method improves the transfection efficiency and thus provides a higher yield of CAR-T cells.

In some embodiments, the method may not be a method of treatment or surgery carried out on the human or animal body. In some embodiments, the method may not be a method for modifying the germ line genetic identity of human beings.

Preferably the method includes the step of mixing the nucleic acid or agent with the at least one amphiphilic construct to form the transfection mixture. Once the nucleic acid or agent is associated with the amphiphilic construct it forms the transfection mixture.

In one embodiment the at least one amphiphilic construct can include or consist of any or any combination of at least one liposomal material or vehicle, at least one pegylated liposomal material or vehicle, a micelle, a construct having a phospholipid bilayer, a cationic polymer, polyethylenimine (PEI) and/or the like.

For example, the cationic polymer can be Turbofect™.

Preferably the nucleic acid is deoxyribonucleic acid (DNA), ribonucleic acid (RNA), or comprises a combination of DNA and RNA (for example, DNA/RNA hybrid oligonucleotides). When the nucleic acid is RNA, it can be mRNA, tRNA, siRNA, miRNA and/or the like.

In one embodiment, the nucleic acid is or includes one or more expression vectors. For example, the one or more expression vectors could be one or more DNA plasmids comprising one or more exogenous genes intended for expression in one or more eukaryotic cells.

In one embodiment, when the agent is nucleic acid, the transfection process results in stable expression, in that the transfected nucleic acid in the transfected cells is continuously expressed and is passed on to daughter cells.

In one embodiment, when the agent is nucleic acid, the transfection process results in transient expression, in that the transfected nucleic acid is only expressed for a relatively short period of time and is not passed on to daughter cells.

Preferably the step of directing pulsed electromagnetic signals takes place at room temperature (such as for example 20° C.) or takes place in an incubator that can be set at temperatures above room temperature or in a patients body (such as for example at 37° C.).

In one embodiment the step of directing pulsed electromagnetic signals takes place for a pre-determined time period. In one example, the time for which the cells receive the pulsed electromagnetic signals is approximately 15 minutes or up to 15 minutes when directed at the transfection mixture in step a) prior to creating the transfection complex. However, it will be appreciated that longer or shorter time periods could be used if required.

In one embodiment the pre-determined time period for which the cells receive the pulsed electromagnetic signals is approximately at or between approximately 1-4 hours when directed at the transfection complex in step c) to form the transfected cells and/or after the transfection step, and further preferably approximately 3-4 hours. However, it will be appreciated that longer or shorter time periods could be used if required. For example, in one embodiment the pre-determined time period can be up to 16 hours, or up to 24 hours.

In one embodiment the transfection is reverse transfection (i.e. the eukaryotic cells are introduced into the transfection mixture).

In one embodiment the transfection is forwards transfection (i.e. the transfection mixture is introduced into the eukaryotic cells).

Preferably the pulsed electromagnetic signals are generated by one or more electronic devices.

Preferably the one or more electronic devices include transmission means for generating and/or transmitting the pulsed electromagnetic signals therefrom in use.

Preferably the transmission means includes one or more electronic transmission chips, the one or more electronic transmission chips arranged to generate, emit and/or transmit one or more pulsed electromagnetic signals in use.

In one embodiment reference to the transmission means or one or more electronic transmission chips could include one or more transmitters, at least one transmitter and at least one receiver, or one or more transceivers. Thus, in one example, the pulsed electromagnetic signals could be transmitted from a central location or a master transmitter and could be received by one or more remote and/or slave receivers and/or transceivers for subsequent re-transmission or emission therefrom.

In one embodiment the electronic device has a single transmission means or electronic transmission chip. Such a single transmission means or electronic transmission chip is sufficient to provide a pulsed electromagnetic signal to a tissue culture plate in one example. In one exemplary embodiment, a single transmission means or electronic transmission chip is provided attached or integrated into a bioreactor containing one or more suspended cells. Such a bioreactor operates by stirring the suspension with a stirrer, and as such the cells suspended, typically in media, will pass by the transmission means or electronic transmission chip and thus be exposed to the pulsed electromagnetic signal of the present invention.

In one embodiment the electronic device has two or more transmission means or electronic transmission chips. Preferably the two or more transmission means or electronic transmission chips are arranged a pre-determined spaced distance apart from each other in the electronic device.

Preferably the pre-determined spaced distance apart is such so as to provide one or more items or material being pulsed with the electromagnetic pulsed signals sufficient signal strength to achieve a desired effect (i.e. of increasing transfection efficiency) and/or to provide an even or substantially even distribution of electromagnetic radiation/signals in use.

Preferably the electronic device has a plurality of transmission means or electronic transmission chips arranged in a pre-determined pattern and/or array.

Whilst a single transmission means or electronic transmission chip is sufficient to provide the advantageous properties of the invention, it has been found that having a plurality of transmission means or electronic transmission chips allows the pulsed electromagnetic signal to be delivered across a broader range of surface areas whilst still maintaining a maximal effect. Applicants have found that having a transmission means or electronic transmission chip evenly distributed such that there is at least one chip per 18.5 cm² provides sufficient coverage for the optimal effect.

In some embodiments, the apparatus comprises one or more transmission means or electronic transmission chips. In some embodiments, the apparatus comprises 2, 3, 4, 5, 6, 7, 8, 9, or 10 or more transmission means or electronic transmission chips.

In some embodiments, there is one transmission means or electronic transmission chip per approximately 105 to 115 cm² of a surface of the housing of the apparatus or a surface of an item as defined herein, and preferably approximately 110 cm² of a surface of the housing of the apparatus or a surface of an item as defined herein.

In some embodiments, there is one transmission means or electronic transmission chip per approximately 50 to 60 cm² of a surface of the housing of the apparatus or a surface of an item as defined herein, and preferably approximately 55 cm² of a surface of the housing of the apparatus or a surface of an item as defined herein.

In some embodiments there is one transmission means or electronic transmission chip per approximately 25 to 30 cm² of a surface of the housing of the apparatus or a surface of an item as defined herein, and preferably approximately 27.5 cm² of a surface of the housing of the apparatus or a surface of an item as defined herein.

In some embodiments there is one transmission means or electronic transmission chip per approximately 15 to 20 cm² of a surface of the housing of the apparatus or a surface of an item as defined herein, and preferably approximately 18.5 cm² of a surface of the housing of the apparatus or a surface of an item as defined herein.

In some embodiments, there is one transmission means or electronic transmission chip per approximately 10 to 15 cm² of a surface of the housing of the apparatus or a surface of an item as defined herein, and preferably approximately 12.2 cm² of a surface of the housing of the apparatus or a surface of an item as defined herein.

The items as defined herein preferably comprise cell culture plates, flasks, roller bottles, and other vessels known to the skilled person. For example, standard laboratory microplates as defined below, T25, T75, T125, T175, T225, and larger cell culture plates. The one or more transmission means or electronic transmission chips are set a pre-determined space apart according to the surface area of such vessels placed on the device in use, and/or based upon a surface of the housing of the apparatus.

In an exemplary embodiment, six transmission means or electronic transmission chips are provided in the apparatus upon which a standard laboratory microplate is positioned. These standard laboratory microplates are provided as 6-well, 12-well, 24-well, 48-well, 96-well, 384-well, and 1536 well plates (and above). These microplates are generally of a standardized size, with dimensions of approximately 128 mm in length by 85 mm in width, thus giving the plate a surface area of approximately 110 cm². Thus, in the exemplary embodiment, the 6 transmission means or electronic transmission chips can be evenly spaced to provide an optimal pre-determined space for providing any of these plate types with a pulsed electromagnetic signal according to the present invention. In one example, the electronic device includes six transmission means or electronic transmission chips. Preferably the six transmission means or electronic transmission chips are arranged a pre-determined distance apart from each other such that when a 24 well plate is located in, on or relative to the electronic device in use, each transmission means or chip is able to emit sufficient strength electromagnetic signals and/or is directed to 4 wells of the plate.

Further preferably the transmission means or transmission chip is located adjacent to the 4 wells of the 24 well plate in a central or substantially central position.

In one embodiment, where more than one transmission means or electronic transmission chip is required, the spacing of the plurality of transmission means or electronic transmission chips must be optimised. In order to achieve an optimal pre-determined space between each transmission means or electronic transmission chips, the transmission means or electronic transmission chips should be positioned at a distance equal or substantially equal to half the wavelength of the electromagnetic radiation frequency being used. Preferably this distance should be considered to be relevant in any plane of orientation or two or more transmission means or electronic transmission chips being used together as part of the apparatus. For example, if the wavelength is 12.4 cm, the transmission chips should be placed approximately 6.2 cm apart to produce an optimal electromagnetic field when in use.

In one example, the pre-determined spaced distance=wavelength/2.

In one example, the pre-determined spaced distance in the X-axis and/or Y-axis is half the wavelength between each transmission means or electronic transmission chip in an evenly spaced grid. Such an arrangement minimises the risk of destructive interference.

In one embodiment the electronic device includes a housing and the one or more transmission means or transmission chips are located in said housing.

Preferably the housing includes at least one flat or planar surfaces to allow the housing to be located in a stable manner with respect to the one more items receiving the pulsed electromagnetic signals in use. Alternatively, the housing can include one or more curved or non-planar surfaces to allow the housing to be located in a stable manner with respect to one or more items receiving the pulsed electromagnetic signals in use.

In one example, at least one surface of the housing includes one or more recesses for the location of the one or more items receiving the pulsed electromagnetic signals in use.

In one example, the electronic device is referred to as a transfection plate for use in a laboratory.

In one embodiment the housing includes a base surface for allowing the housing to be supported directly or indirectly on a surface in use. Further preferably the housing includes an upper surface opposite to the base surface. Preferably the upper surface is the surface on which the one or more items receiving the pulsed electromagnetic signals can be positioned in use.

In one example, the one or more items can be cell culture plates or flasks known to the person skilled in the art in which eukaryotic cells may be cultured.

In one embodiment the electronic device and/or housing is attachable to an external surface of a container, reactor vessel and/or the like. For example, the electronic device and/or housing can be attachable via one or more attachment means or device including any or any combination of one or more screws, nuts and bolts, magnets, ties, clips, straps, inter-engaging members, adhesive, welding and/or the like.

Preferably the upper surface of the housing and/or the distance between the transmission means and the one or more items receiving the pulsed electromagnetic signals when located on, in or relative to the housing or electronic device in use is approximately 25 cm or less, 20 cm or less, 15 cm or less, 10 cm or less or 5 cm or less. Further preferably the distance is approximately 1 cm.

Preferably the pulsed electromagnetic signals are provided in a pre-determined sequence of pulses.

In one embodiment the electronic device is arranged to transmit the pulsed electromagnetic signals at a frequency in the range of approximately 2.2-2.6 GHz and, further preferably the pulsed electromagnetic signals are transmitted at a frequency of approximately 2.4 GHz+/−50 MHz or more preferably 2.45 GHz+/−50 MHz.

In one embodiment the electronic device is arranged to transmit the pulsed electromagnetic signals at a frequency within the range of the industrial, scientific and medical radio frequency band (ISM band) of 2.4 to 2.4835 GHz, preferably 2.45 GHz+/−50 MHz.

Preferably the pulsed electromagnetic signals are pulsed at a frequency of approximately 50 Hz or less, further preferably approximately 25 Hz or less, and yet further preferably approximately 15 Hz or less.

Preferably each pulse of the pulsed electromagnetic signals lasts for between approximately 1 ms-20 ms. Further preferably each pulse lasts for approximately 1 ms.

Preferably the time period between pulses (also referred to as the “rest period” or “relaxation period”) is approximately 66 ms or less.

Preferably the duty cycle of the pulsed electromagnetic signals is less than 2%.

In one embodiment the transmission power provided by each transmission means or chip in the electronic device is 2 dBm-+4 dBm, approximately 1 mW, approximately 2 mW or approximately 2.5119 mW.

In one embodiment the pre-determined frequency of the pulsed electromagnetic signals is approximately 2.2-2.6 GHz, 2.4 GHz+/−50 MHz or 2.45 GHz+/−50 MHz, the pre-determined pulse rate is approximately 15 Hz or has a duty cycle of less than 2%, and the pre-determined power is +2 dBm-+4 dBm, 1 mW, 2 mW or 2.5119 mW.

Without wishing to be bound by theory, use of electromagnetic waves or signals used in the apparatus or methods of the present invention are thought to be sufficient to rotate H₂O periodically around its dipole with relatively long rest or relaxation periods. The periodic rotation of H₂O is thought to interrupt hydrogen bonding in the phospholipid bilayer and/or amphiphilic constructs. This periodic or intermittent low energy perturbation of the cell membranes is thus thought to stimulate increased interaction with some molecules, cell membranes and/or amphiphilic constructs and their environment, such as for example, the nucleic acid or agent associated with the amphiphilic construct. This is thought to enhance the transport of agents across the cell membrane, leading to an increased uptake of the one or more agents such as nucleic acids, peptides, small molecules and other agents by the one or more eukaryotic cells. Thus, it can be seen that the transfection and/or intra-cellular delivery process according to the present invention can be significantly improved using very low energy electromagnetic waves or signals. The relatively long rest or relaxation period between the pulses of the pulsed electromagnetic signals is thought to be sufficient to maintain cellular integrity. Thus, in the context of the present invention, the use of pulsed electromagnetic signals, waves or fields, is thought to provide an improved transport of molecules across the cell membrane, leading to a more efficient transfection and/or intracellular delivery of agents as defined earlier.

Preferably the pulsed electromagnetic signals are transmitted using Gaussian Frequency Shift Keying (GFSK) between 0.45 and 0.55.

Preferably the pulsed electromagnetic signals are radio frequency (RF) data signals.

Preferably the pulsed electromagnetic signals is a digital sequence of pulsed electromagnetic signals.

Preferably the radio frequency signals utilize the Bluetooth LE (BLE) protocols advertising feature. Preferably the advertising RF signals are on channels 37, 38 and 39 corresponding to frequencies 2402 MHz, 2426 MHz, 2480 MHz respectively.

Preferably the pulsed electromagnetic signals are directed towards aqueous media consisting of or including the transfection mixture, transfection complex and/or a post transfection complex.

In one embodiment the electronic device includes power supply means for supplying electrical power to the device in use. Preferably the power supply means includes a mains electrical power supply, one or more batteries, power cells, one or more rechargeable batteries, electrical generator means and/or the like.

In one embodiment the electronic device includes control means for controlling operation of the electronic device and/or transmission means in use.

In one embodiment the electronic device includes one or more circuit boards. Preferably the transmission means can be provided on the one or more circuit boards, typically in the form of an integrated circuit, and/or other components, such as for example memory means, are located.

In one embodiment the electronic device includes memory means, such as a memory device, data storage device and/or the like.

Preferably the other components of the electronic device includes one or more components required for the selective operation of the apparatus and, when active, the controlled operation of the same to generate the pulsed electromagnetic signals. For example, user selection means can be provided on the device to allow user selection of one or more conditions, operation and/or one or more parameters of the device in use; display means to display one or more settings, options for selection and/or the like.

In one embodiment the said further components or power supply means include one or more power cells and the same may all be contained within the housing.

In one embodiment the housing of the electronic device is provided in a form which allows the same to be engaged with and/or located with respect to a container in which the material and/or one or more items which is to be exposed to the electromagnetic signals is located in use.

In one embodiment the control means includes an option to allow the user to select any or any combination of the signal frequency, signal strength, signal power, signal pulse rate, time period of signal pulsing, and/or the like of the said pulsed electromagnetic signals. In one embodiment the selection of the frequency, strength, power, pulse rate, time period of pulsing, other parameters and/or the like may be made with respect to the particular form of the material and/or one or more items which is to be exposed to the pulsed electromagnetic signals in use, the quantity of said material, the dimensions of the container with respect to which the apparatus is located for use and/or other parameters.

It has been found the cells exposed to pulsed signals like those of the present invention provide a uniform or substantially uniform distribution or dispersion of cells during transfection in vitro, in contrast to transfection where no pulsed technology is used and clumping of cells has been observed.

According to an aspect of the present invention there is provided apparatus for providing improving transfection efficiency in eukaryotic cells, said apparatus including a housing, transmission means located in said housing and arranged to transmit pulsed electromagnetic signals provided at any or any combination of a pre-determined frequency, at a pre-determined pulse rate, or a pre-determined power in use, control means for controlling operation of at least the transmission means in use, and power supply means for providing electrical power to the transmission means and/or control means in use.

Preferably the one or more pre-determined parameters of the apparatus can be pre-set by the manufacturer of the apparatus and/or can be user selectable depending on the users requirements.

Preferably the control means are used to allow user selection of one or more of the user selectable pre-determined parameters.

In one embodiment the apparatus is arranged to be directly or indirectly worn on or adjacent the skin of a person in order to allow the pulsed electromagnetic signals to be directed towards an area of the persons body in use for improving a transfection process in the persons body. In this embodiment the apparatus is preferably a wearable device.

In one embodiment, attachment means can be provided on and/or associated with the apparatus to allow detachable attachment to, or relative to, the exterior of a users skin or body, the interior and/or exterior of a garment or item worn by the user in use and/or the like for improving a transfection process taking place in the persons body.

In an exemplary embodiment, the apparatus is a wearable device, for example an armband, and the armband is placed directly on the site of injection of, for example, a DNA or RNA vaccine administered to a patient.

In an exemplary embodiment, there is a method for administration of a vaccine comprising injecting the vaccine into a subject, and then placing the apparatus of the invention on the site of injection and providing pulsed electromagnetic signals according to the present invention to the injection site.

Preferably apparatus and/or the transmission means or one or more electronic transmission chips are arranged in the apparatus so that the pulsed electromagnetic signals are directed to the users skin or body in use. For example, the pulsed electromagnetic signals can be directed through a first surface of the housing, and said first surface is arranged to be in direct or indirect contact with a users skin.

In one embodiment the apparatus is arranged to be implantable into a persons body. For example, the apparatus could be implanted at a site in the persons body requiring treatment. In this embodiment the apparatus is preferably an implant.

Preferably at least the outer casing of the apparatus is coated and/or formed from a material suitable for implantation into a persons body.

Preferably the attachment means includes any or any combination of a one or more straps, ties, necklaces, pendants, belts, bracelets, clips, keyrings, lanyards, VELCRO® (hook and loop fastening), press studs, buttons, button holes, adhesive, plaster, sutures, clips, bio-compatible adhesives and/or the like.

In one embodiment, the apparatus is provided with at least one holding means or reservoir for holding or containing the transfection mixture which is to be transfected into a person in use respectively.

Preferably the holding means or reservoir is arranged on the apparatus such that it is locatable on and/or adjacent to a persons skin in use. The pulsed electromagnetic signals can be directed at one or more parts of a persons body to help improve the absorption and/or transfection of the agent through the persons skin and into one or more cells of the person.

In one embodiment, it is thought that the direction of pulsed electromagnetic signals to a users skin modifies the permeability of the users skin to allow increased and/or improved take up of the transfection mixture in use. Typically, modification of the permeability of the skin occurs at least for the time period during which the pulsed electromagnetic signals are directed towards a users skin. Typically, the modification of the permeability of the users skin remains, but diminishes over time once the pulsed electromagnetic signal emission has stopped.

In one embodiment the strength and range of the pulsed electromagnetic signals is sufficient, when the housing the electronic device is located with respect to a portion of the users skin, for the pulsed electromagnetic signals to pass through the skin into the users body, and preferably at least adjacent an inner area immediately adjacent said users skin portion.

According to one aspect of the present invention there is provided a method of increasing transfection efficiency in eukaryotic cells and/or apparatus for increasing transfection efficiency in eukaryotic cells.

According to a further aspect of the present invention there is provided a method of increasing protein expression in transfected eukaryotic cells and/or apparatus for increasing protein expression in transfected eukaryotic cells.

According to one aspect of the present invention there is provided a method for providing gene therapy in vivo, said method comprising the steps of:
a) providing a transfection mixture including an agent associated with at least one amphiphilic construct suitable for transfection;
b) introducing or injecting the transfection mixture into a patient to allow transfection of one or more cells of the patient in vivo with the transfection mixture;
   characterised in that the method includes the step of directing pulsed electromagnetic signals provided at any or any combination of a pre-determined frequency, at a pre-determined pulse rate, and for at a pre-determined power at the transfection reagent at step a) prior to directing or injecting the transfection mixture, at the patient during the directing or injecting of the transfection mixture into the patient in step b) and/or at the patient after the transfection step b).

Preferably the method of introducing the transfection mixture into the patient includes orally, transdermally, sub-cutaneously and/or the like.

According to one aspect of the present invention there is provided a method for providing gene therapy in vitro, said method comprising the steps of:
a) providing a transfection mixture including an agent associated with at least one amphiphilic construct suitable for transfection;
b) adding the transfection mixture to one or more eukaryotic cells, taken from a patient prior to the method, to form a transfection complex;
c) transfecting the transfection complex to form one or more transfected cells;
characterised in that the method includes the step of directing pulsed electromagnetic signals provided at any or any combination of a pre-determined frequency, at a pre-determined pulse rate, and at a pre-determined power, at the transfection mixture at step a) prior to creating the transfection complex, at the transfection complex in step b), at the transfection complex in step c) and/or at the transfected cell complex after the transfection step c).

According to a further aspect of the present invention there is provided a method of improving transfection efficiency in eukaryotic cells, said method including the steps of:
a) providing a transfection mixture including nucleic acid associated with at least one amphiphilic construct suitable for transfection;
b) adding the transfection mixture to one or more eukaryotic cells to form a transfection complex;
c) allowing the transfection complex to undergo a transfection process to form one or more transfected cells;
characterised in that the method includes the step of directing pulsed electromagnetic signals provided at any or any combination of a pre-determined frequency, at a pre-determined pulse rate, and at a pre-determined power, at the transfection mixture at step a) prior to creating the transfection complex, at the transfection complex in step b), at the transfection complex in step c) and/or at the transfected cells complex after the transfection step c).

Once the patients cells have been transfected according to the method, they can then be optionally re-introduced back into the patient or another patient as required.

According to an aspect of the present invention there is provided apparatus for assisting in the provision of gene therapy in eukaryotic cells, said apparatus including a housing, transmission means located in said housing and arranged to transmit pulsed electromagnetic signals provided at any or any combination of a pre-determined frequency, at a pre-determined pulse rate, or a pre-determined power in use, control means for controlling operation of at least the transmission means in use, and power supply means for providing electrical power to the transmission means and/or control means in use.

According to a further aspect of the present invention there is provided a method of altering gene and/or protein expression, said method comprising the steps of:
providing one or more eukaryotic cells
characterised in that the method includes the step of directing pulsed electromagnetic signals provided at any or any combination of a pre-determined frequency, at a pre-determined pulse rate, and at a pre-determined power, at the eukaryotic cells to alter the gene expression and/or protein expression in said one or more eukaryotic cells.

In one embodiment the method kills cancer cells and increases DNA repair in healthy cells and tissue.

In one embodiment the apparatus is implantable into a patient, such as for example in a region at or adjacent cancerous tissue, to treat the cancerous tissue. This method may be useful where cancerous tissue is more distant from the patients skin.

In one embodiment the apparatus is worn by a patient at or adjacent the patients skin and could be used to deliver one or more pharmaceutical agents or drugs to cancerous tissue, such as for example located in the vicinity of a sub-dermal tumour, such as a melanoma, and/or to treat a virus.

Thus, in one embodiment, the apparatus can be used to deliver pulsed electromagnetic signals through a patients skin to interact directly with the DNA of cells to promote the apoptosis, cell of cancerous cells and/or assist in creating healthy cells to repair DNA damage.

In one embodiment the apparatus is used to deliver pulsed electromagnetic signals through a patients skin to provide an anti-viral effect.

In one aspect of the present invention there is provided a cell or progeny thereof produced using any one of the methods defined herein.

It is to be noted that reference to an improvement in transfection efficiency herein refers to an increase in the number of cells transfected and an increase or maintenance of the cell viability following a transfection process.

It will be appreciated that the present invention can be used in a laboratory based environment or can be upscaled to be used in an industrial level environment.

Specific embodiments of the invention are now described with reference to the accompanying drawings; wherein

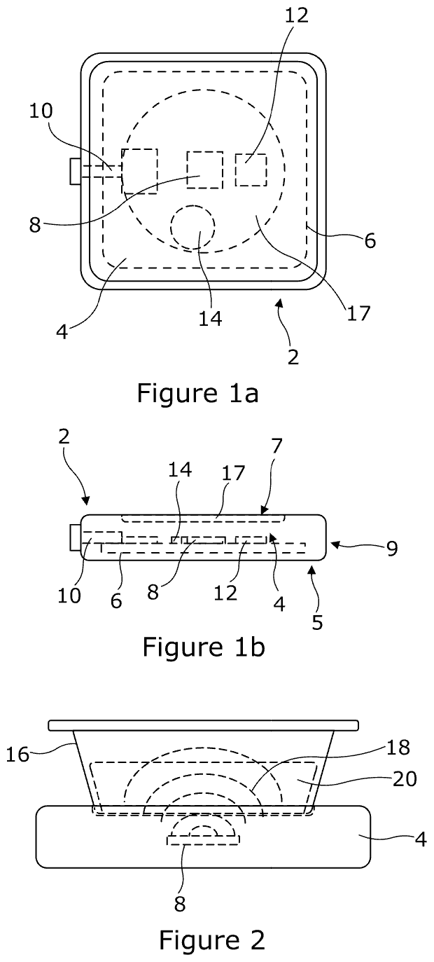

FIGS. 1a and b illustrate apparatus in accordance with one embodiment of the invention in which the electronic device includes one transmitter chip;

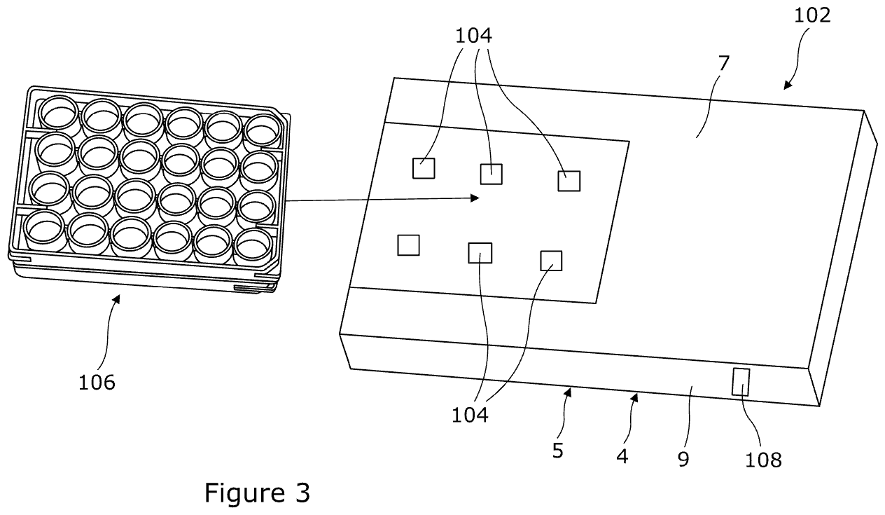

FIG. 2 illustrates the apparatus of FIG. 1 in use to perform the method in accordance with the invention in one embodiment;

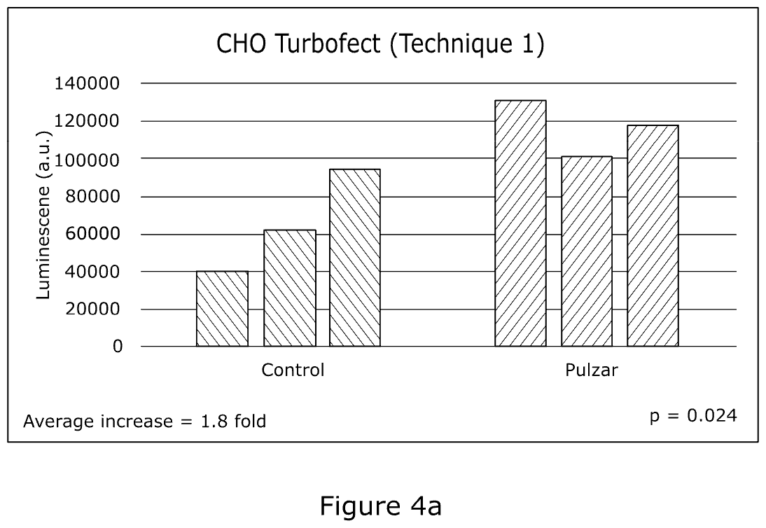

FIG. 3 illustrates apparatus in one embodiment of the present invention in which the electronic device includes an array of 6 transmitter chips, together with an example of a twenty-four well plate that can be used with the electronic device in one example;

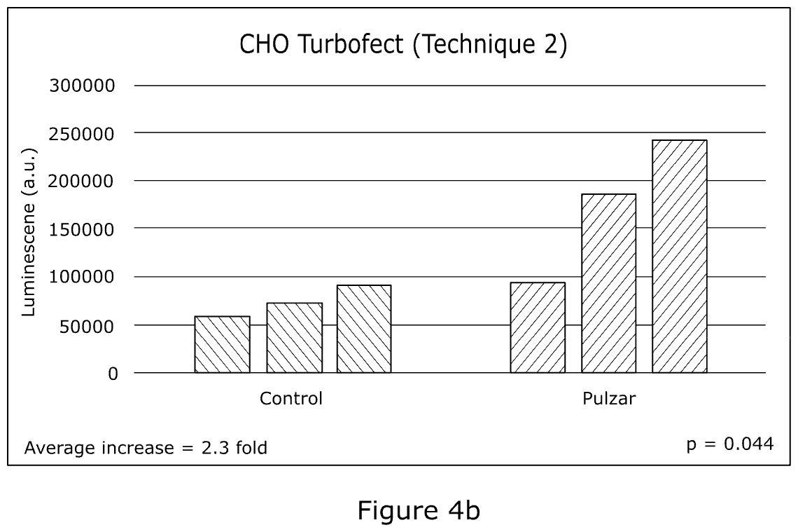

FIG. 4a shows the results of transfection of adherent CHO K1 cells using a DNA plasmid associated with a Turbofect amphiphilic construct, where pulsed technology comprising a single electronic transmitter chip was used according to an embodiment the present invention;

FIG. 4b shows the results of transfection of adherent CHO K1 cells using a DNA plasmid associated with a Turbofect amphiphilic construct, where pulsed technology comprising six electronic transmitter chips were used according to an embodiment of the present invention;

FIG. 4c shows the results of transfection of adherent HCT 116 cells using a DNA plasmid associated with a Turbofect amphiphilic construct, where pulsed technology was used according to an embodiment the present invention;

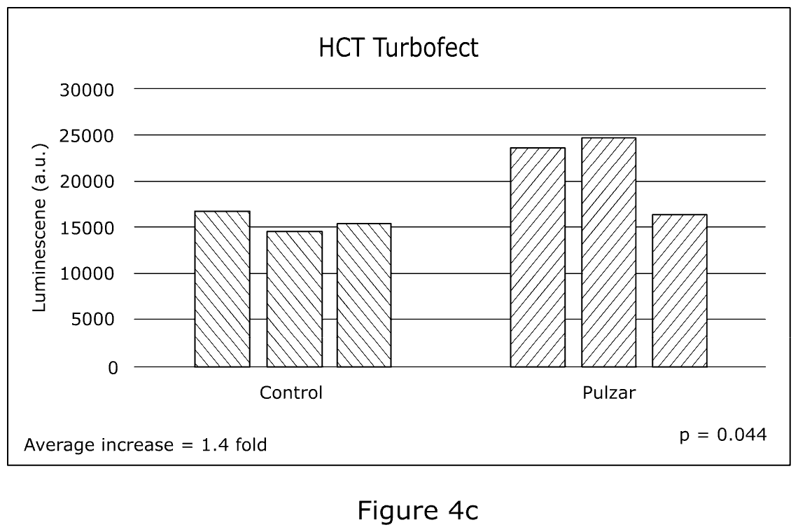

FIG. 5a shows the results of transfection in HCT 116 cells using a DNA plasmid with an IGFBP3 promoter and associated with a PEI amphiphilic construct using pulsed technology of the present invention;

FIG. 5b shows the results of transfection of HCT 116 cells using a DNA plasmid with a SV40 promoter and associated with a PEI amphiphilic construct using pulsed technology of the present invention;

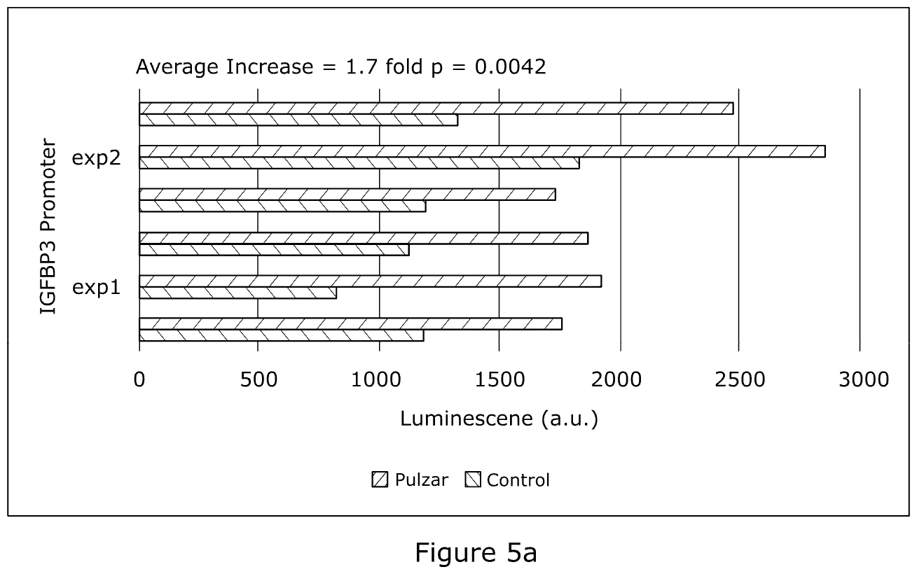

FIG. 6 is a graph showing the results of transfection in suspended HEK 293 Freestyle cells using a Green Fluorescent Protein (GFP) containing plasmid associated with a PEI amphiphilic construct using pulsed technology of the present invention;

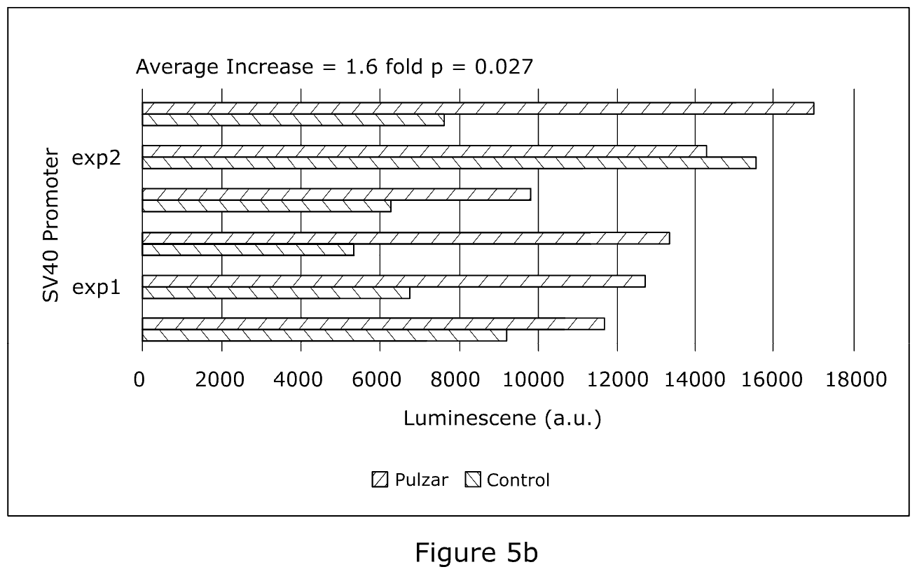

FIG. 7 is a graph showing further results of transfection in suspended HEK 293 Freestyle cells using a Green Fluorescent Protein (GFP) containing plasmid associated with a PEI amphiphilic construct using pulsed technology of the present invention;

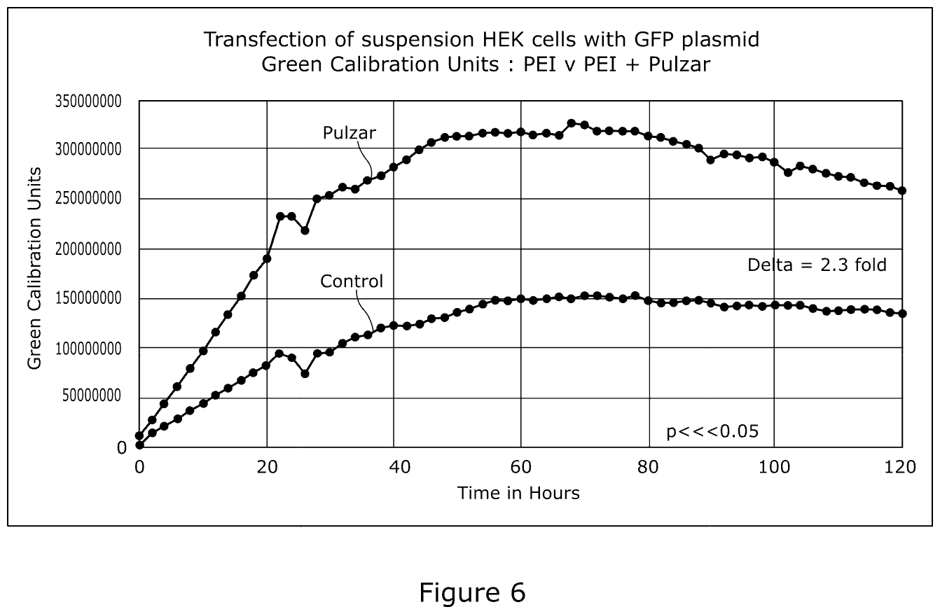

FIG. 8 is a graph showing results of transfection in suspension Jurkat E6 cells using a DNA plasmid associated with a TransIT2020 amphiphilic construct using pulsed technology of the present invention;

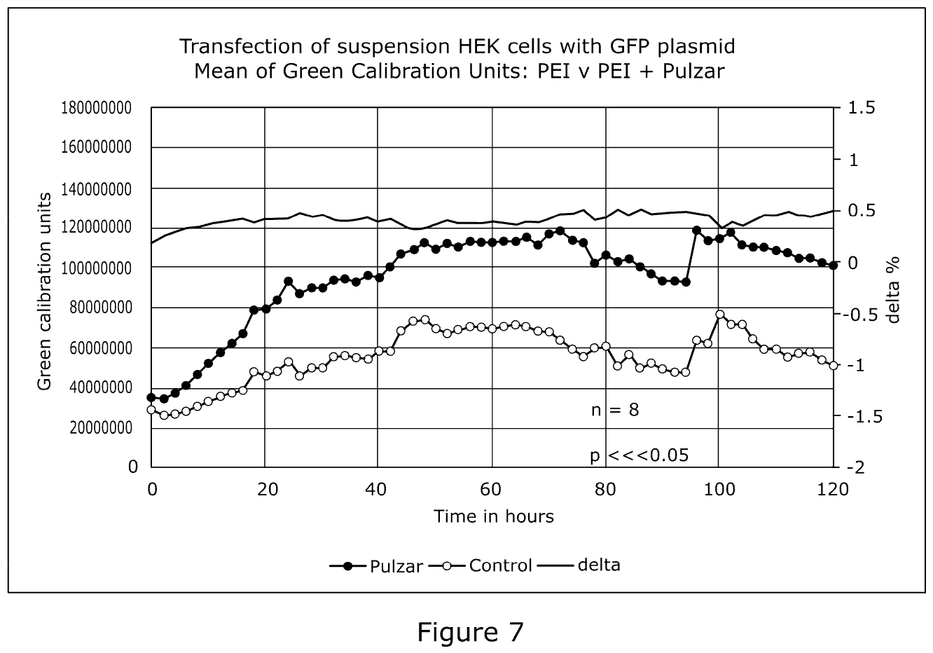

FIGS. 9a and 9b illustrate views of apparatus in accordance with an embodiment of the present invention;

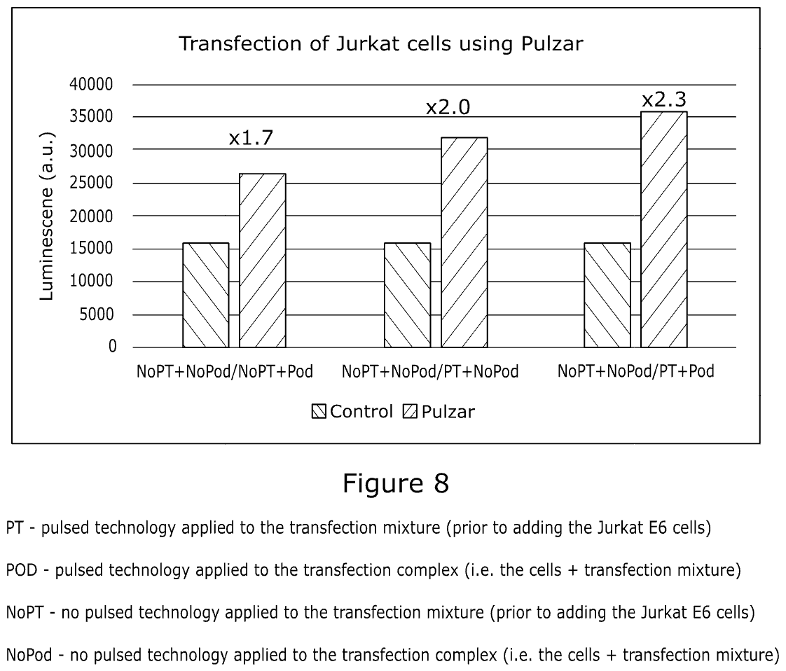

FIGS. 10a and 10b illustrate views of apparatus in accordance with a further embodiment of the present invention; and

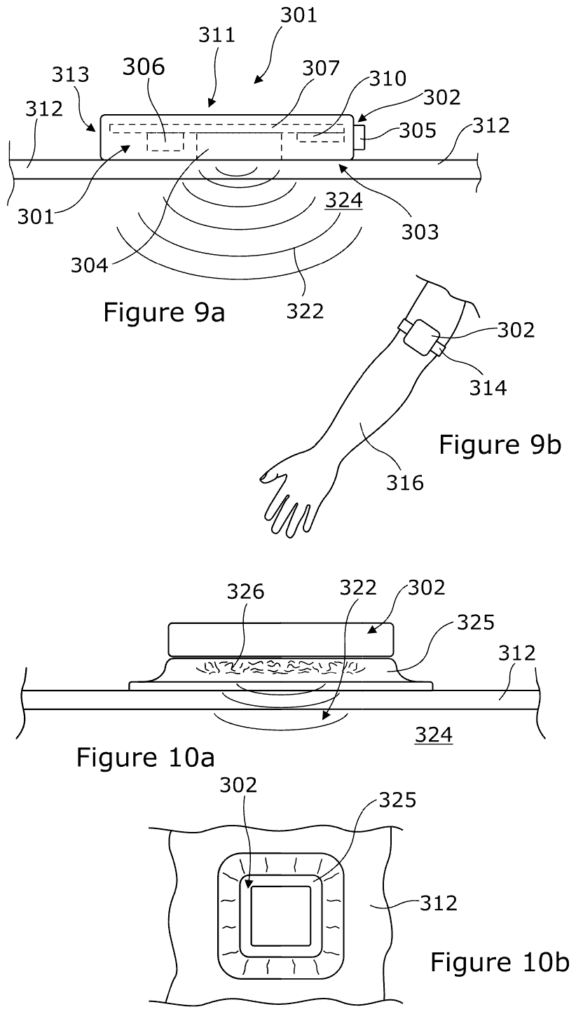

FIG. 11 illustrates a further embodiment of the present invention;

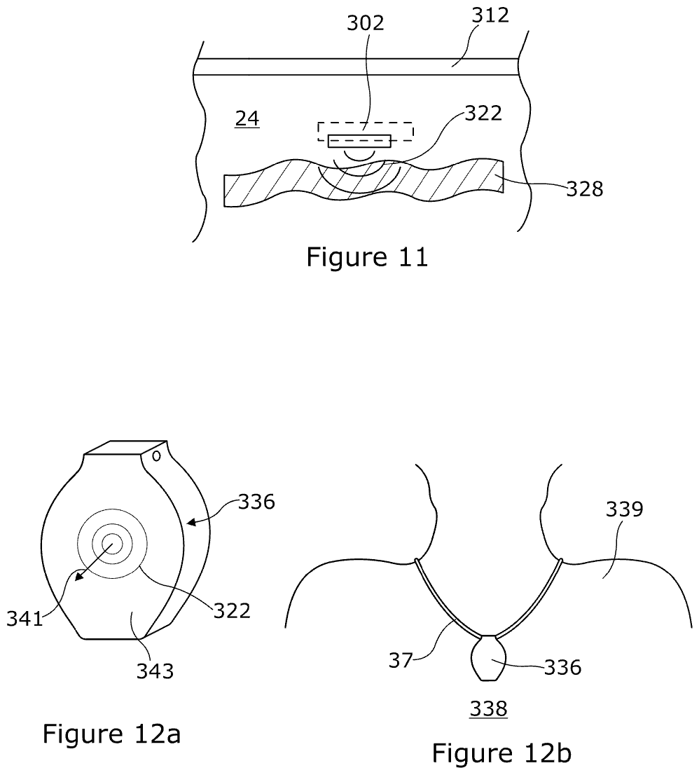

FIGS. 12a and 12b illustrate elevations of a yet further embodiment of the present invention;

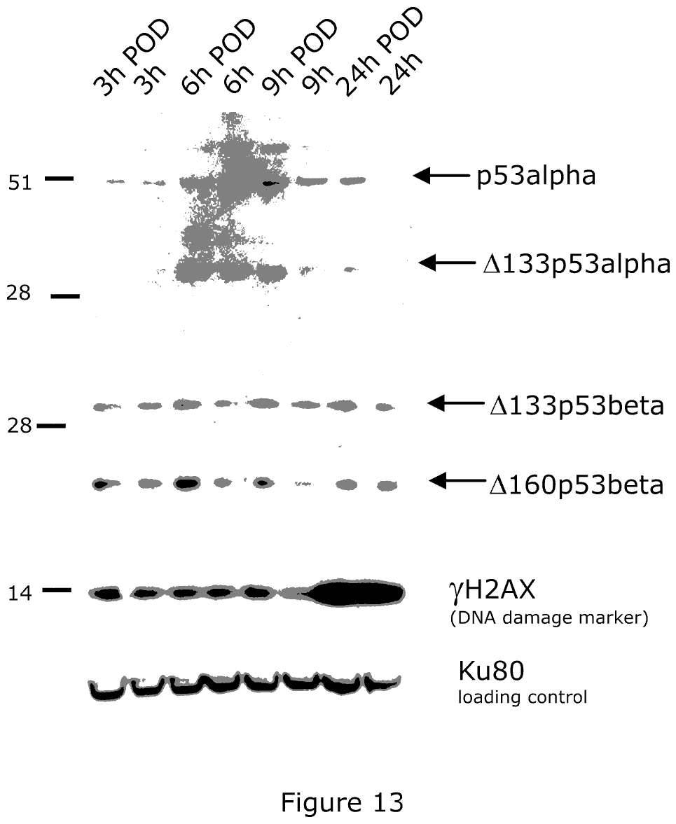

FIG. 13 shows a western blot from an experiment in the applicants co-pending patent application providing support for the claims of the present invention;

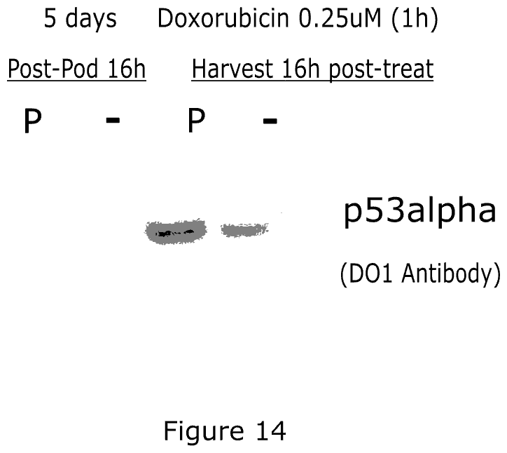

FIG. 14 shows a further western blot from an experiment in the applicants co-pending patent application providing support for the claims of the present invention.

With reference to FIGS. 1a, 1b and 2, there is illustrated apparatus 2 for performing the method of the present invention of improving transfection efficiency in eukaryotic cells in one embodiment.

The apparatus 2 is in the form of an electronic device capable of emitting pulsed electromagnetic signals at a pre-determined frequency, at a pre-determined pulse rate, at a pre-determined power level and for a pre-determined period of time. The pre-determined parameters can be pre-set by the manufacturer or can be user selectable as required. The technology used in the apparatus is referred to hereinafter as the “pulsed technology according to the present invention”.

Apparatus 2 includes a housing 4. In this particular example, the housing 4 is in the form of a laboratory transfection plate, and includes a base surface 5, an upper surface 7 opposite to base surface 5, and one or more side walls 9 located between the upper and base surfaces 5,7.

Within the interior of housing 4 there is provided a circuit board 6 with an integrated circuit 8 configured and interconnected thereon to generate pulsed electromagnetic signals when operational. Control means in the form of a control unit 10 are provided to allow the selective operation of the apparatus 2. A memory device 12 is provided to allow data, one or more operating parameters, software and/or the like to be stored and retrieved when necessary. The control unit preferably includes micro-processing means to allow processing of data and/or the like.

The apparatus 2 typically also includes one or more power cells 14 to provide electrical power to the apparatus. A rechargeable facility can also optionally be provided to allow the power cells 14 to be recharged from a remote power source rather than having to be replaced.

It will be appreciated that the housing 4 may be provided in any suitable form for its intended use and can be provided with engagement means to allow the same to be located with, for example an interior or exterior of a container in which the cells to be treated are located. Alternatively, the housing may be formed as part of a container in which the cells to be treated are located. Alternatively still, the upper surface 7 can provide a planar or flat surface on which a container in which the cells are to be treated or located can be placed. Yet further still, a recess 17 could be defined in the upper surface 7 of the housing for stably supporting the placement of a container 16 in the form of, for example, a culture flask, petri dish or other culture container, so that the housing 4 is located underneath the container 16 and the container 16 is supported in the recess 17.

The integrated circuit 8 includes an electronic transmission chip that is arranged to emit the pulsed electromagnetic signals from the apparatus 2 in use. More particularly, in one embodiment of the present invention, the electronic transmission chip is arranged such that it is spaced less than 5 cm from the container 16 located in recess 17 in use, and preferably approximately 1 cm. This allows the electromagnetic signals emitted from the chip to be directed to the cells located within the container 16 in use.

The apparatus of the present invention is designed to be used at room temperature (i.e. approximately 20° C.), in temperatures colder than room temperature, such as for example in a refrigeration unit, and/or can be used at temperatures above room temperature, such as for example in an incubator unit.

In one embodiment, the control unit 10 is programmed to control the transmission chip to allow it to emit pulsed electromagnetic signals at a frequency of 2.45 GHz+/−50 MHz, at a pulsed frequency of 15 Hz and at a power of approximately 2 mW. It will be appreciated that the parameters associated with the pulsed electromagnetic signals can be adjusted and/or be user selectable as required. For example, the time for which the pulsed electromagnetic signals are emitted can be selected by the user if required. In addition, the power can be adjusted, although it typically remains in the milliwatt range so as to avoid over energising the cells contained within the container 16 in use. In one example, the pulsed signals last for 1 ms and the rest period between signals is 66 ms. This provides a duty cycle of less than 2%.

In one example, the electromagnetic signals are RF signals using the Bluetooth LE protocols advertising feature and are transmitted using GFSK between 0.45 and 0.55.

However, it should be noted that any frequency transmission in the Industrial, Medical and Scientific frequency bands (i.e. 2.4 to 2.4835 GHz, preferably 2.45 GHz+/−50 MHz) could be possible by the electronic apparatus in use.

Referring to FIG. 3, there is illustrated a further example of apparatus 102 for providing the pulsed electromagnetic signals according to a further embodiment. Whereas, FIGS. 1a-1b show apparatus comprising a single electronic chip for transmission of the pulsed electromagnetic signals, FIG. 3 shows apparatus 102 that as an array of six electronic chips 104 for transmission of the pulsed electromagnetic signals. The same reference numerals are used to describe the same features as in FIGS. 1a-1b. Although FIG. 3 shows the electronic chips 104 as being on top of the apparatus 102, this is just shown like this for clarity and the chips 104 are actually contained within the apparatus 102.

The six electronic chips 104 are provided a spaced distance apart in the apparatus 102. The spacing between the chips can be any required distance but, in one example, the chips are spaced apart such that when a 24 well cell plate 106 is located on upper surface 7 of the apparatus in use, one transmission chip 104 is located centrally of four of the wells. Thus, each electronic chip 102 directs pulsed electromagnetic signals to 4 wells per 24 well cell plate. An on/off operational switch 108 is provided on the apparatus 102 to move the apparatus between on and off conditions in use.

In accordance with the present invention, the apparatus as described above can be used to provide pulsed electromagnetic signals directed towards reagents and/or cells involved at one or more different stages of a transfection process. The apparatus can also be used to direct pulsed electromagnetic signals to transfected or non-transfected cells to enhance cellular protein expression. The pulsed technology of the present invention has wide and different application uses, such as gene therapy, cell transfection and/or the like as previously described.

The Applicants have undertaken experiments to show that when an agent in the form of nucleic acid, such as DNA, RNA, DNA plasmids and the like, is provided in association with an amphiphilic construct, such as a liposome vehicle, and transfected into different types of eukaryotic cells, the use of their inventive pulsed technology at different stages of the transfection process can significantly increase the transfection efficiency process and the protein expression yield.

As a simplified overview, in one example, material comprising a combined dispersion of eukaryotic cells and liposomal formulations of nucleic acid (DNA, RNA or small segments of either) is contained in a suitable container such as a culture vessel, flask or dish which, in one embodiment is located on the apparatus 2, 102 and pulsed electromagnetic signals are emitted from the apparatus and are directed through the wall of the container 16 and into the material 20.

The pulsed technology of the present invention can be used on the transfection mixture prior to transfection taking place, such as for example on the nucleic acid and/or amphiphilic constructs. The pulsed technology of the present invention can also be used, or alternatively be used, on the transfection complex including the transfection mixture and the eukaryotic cells. In addition, or alternatively still, the pulsed technology of the present invention can be used on the cells once transfection has taken place, and/or on eukaryotic cells which have not undergone transfection to increase protein expression in those cells.

In the following experiments used to exemplify the present invention, the same pulsed technology of the present invention was used on the transfection mixture prior to mixing with different eukaryotic cells lines, and/or on the eukaryotic cell lines mixed with the transfection mixture during a transfection process.

The nucleic acid used in the experiments comprised DNA plasmid material including a arginine vasopressin (AVP) promoter, a simian virus 40 (SV40) promoter, or an insulin like growth factor binding protection 3 (IGFBP3) promoter. A cytomegalovirus (Adluc) plasmid, a luciferase control vector (Renilla) plasmid or a Green Fluorescent Protein (GFP) plasmid were also used.

The amphiphilic constructs used in the experiments were either a transfection reagent containing cationic polymer (Turbofect™) (Thermo Fisher, USA), polyethylenimine (PEI) (Fisher Scientific, USA), or TransIT2020 (Mirus Bio, USA).

The cell lines used in the experiments were Chinese Hamster Ovary K1 (CHO) cells (adherent cells) (ATCC, USA-ATCC® CCL-61™), Human Embryonic Kidney (HEK) 293 freestyle cells (suspension cells) (Thermo Fisher, USA), Human Colon Tumour (HCT) 116 cells (adherent cells) (ATCC, USA-ATCC® CCL-247™) or Jurkat E6 (suspension T-cells) (ECACC), UK).

In order to determine the efficiency of the cell transfection process using the above components, the luciferase activity or the amount of green fluorescent protein was measured using suitable equipment.

The DNA plasmid material chosen was complexed with the amphiphilic construct using known techniques to form a transfection mixture. In some experiments this transfection mixture was subjected to the pulsed technology of the present invention. The transfection mixture (with or without being exposed to pulsed technology) was then mixed in a dispersion of one of the mammalian cell lines in a suitable cell culture container to form a transfection complex. This cell culture container was then placed on the apparatus housing of the present invention and subjected to the pulsed technology as previously described for a predetermined period of time. The emission of the pulsed electromagnetic signals was then stopped and the material was allowed to reach equilibrium. In addition, control experiments were also conducted using the same material and mixing requirements identically but in the absence of the pulsed technology of the present invention.

A more detailed description of the methodology used in the experiments, the results and the findings are provided below.

### Methodology

### Experiment 1—Transfection of Adherent CHO K1 and HCT116 Cells Using Adluc and Renilla Plasmids and Using Either PEI or Turbofect as the Amphiphilic Construct

This experiment was undertaken to look at the effect of the pulsed technology of the present invention on the process of transfection in adherent Chinese Hamster Ovary (CHO) K1 cells (ATCC, USA) and HCT116 (Human Colon Cancer Cell Line) (ATCC, USA) using Adluc and Renilla Plasmids in either PEI (Fisher Scientific, USA) or Turbofect (Thermo Fisher, USA) amphiphilic constructs. The pulsed technology was applied to a) the cells and the transfection mixture (the transfection complex) during the transfection process only; and b) the transfection mixture prior to forming a transfection complex with the cells and then to the transfection complex during the transfection process.

Consumables

Opti-MEM™ I Reduced Serum Media (Thermo Fisher, USA)

Dulbeccos Modified Eagle Medium (DMEM) (Thermo Fisher, USA)

Fetal Calf Serum (FCS) (Hyclone, USA)

2×24 Well Plates Nunc (1.9 cm²/well) (Thermo Fisher, USA)

200 ng of AdLuc plasmid/well (Luciferase expressing plasmid/DNA) (made by Dundee University, UK)

2 ng Renilla plasmid/well (Luciferase expressing plasmid/DNA) (made by Dundee University, UK)

Alfa Aesar™ Polyethyleneimine, linear, M.W. 25.00 (PEI) (Fisher Scientific, USA)

Turbofect (Thermo Fisher, USA)

Method Steps

Control—Using PEI

1. 650 μL of Opti-MEM media was mixed with 2.6 μg of AdLuc plasmid and 26 ng of Renilla plasmid in a first tube;

2. 650 μL of Opti-MEM media was mixed with 7.88 μg of PEI in a second tube;

3. The contents of the second tube was mixed in a dropwise manner to the first tube while gently vortexing until a final volume of 1.3 mL mixture was achieved using a Vortex-Genie 2, Model G560E, (Scientific Industries, USA);

4. The transfection mixture was incubated for 15 minutes at room temperature (approx. 20° C.);

5. 100 μL of this incubated transfection mixture was then dispensed into wells labelled A1-A6 on each of the two 24 well plates (Plates 1 and 2). This formed the transfection mixture.

Invention—with Pulsed Technology Using PEI on Transfection Mixture Prior to Transfection Complex being Created

1. Then, steps 1-3 above were repeated but at step 4 the mixture forming the transfection mixture was incubated for 15 minutes at room temperature (approx. 20° C.) by locating the first tube on a pulsed electromagnetic signal device according to the present invention. The pulsed device operates as described above (i.e. pulsed device operated at 2.45 GHz+/−50 MHz, at power 2 mW using a pulsed frequency of 15 Hz).

2. 100 μL of this incubated pulsed transfection mixture was dispensed into wells labelled B1-B6 on each of the two 24 well plates (Plates 1 and 2);

Control—Using Turbofect
1. 650 μL of Opti-MEM media was mixed with 2.6 μg of AdLuc plasmid and 26 ng of Renilla plasmid in a first tube;
2. 13 μL of Turbofect was added and mixed by vortexing using a Vortex-Genie 2, Model G560E, (Scientific Industries, USA);
3. The transfection mixture was incubated for 15 minutes at room temperature (approx. 20° C.)
4. 100 μL of this incubated transfection mixture was dispensed into wells labelled C1-C6 on each of the two 24 well plates (Plates 1 and 2);
   Invention—Using Turbofect with Pulsed Technology on Transfection Mixture Prior to the Transfection Complex being Created
1. Steps 1-2 above were repeated for the Turbofect Control. At step 3 the transfection mixture was incubated for 15 minutes at room temperature (approx. 20° C.) by locating the first tube on a pulsed electromagnetic signal device according to the present invention. The pulsed device operated at 2.45 GHz+/−50 MHz, at power 2 mW using a pulsed frequency of 15 Hz.
2. 100 μL of this incubated pulsed transfection mixture was dispensed into wells labelled D1-D6 on each of the two 24 well plates (Plates 1 and 2);

### Cell Lines Added to Plates 1 and 2

For the Plates 1 and 2, a transfection complex was created by adding either CHO K1 cells or HCT116 cells into each well of the two 24 well plates at 2×10⁴ cells/well and then made up to a final volume of 600 μL of Dulbeccos Modified Eagle Medium (DMEM)+10% Fetal Calf Serum (FCS). In particular, A1-A3, B1-B3, C1-C3 and D1-D3 had CHO K1 cells added; A4-A6, B4-B6, C4-C6 and D4-D6 had HCT 116 Cells added;
Plates 1 and 2 were incubated in an incubator at 37° C., 5% CO² for 3 hours;
In plate 1 there was no pulsed technology given to the transfection complex during the 3 hour incubation stage, whereas plate 2 was subjected to pulsed technology according to the present invention for 3 hours during the incubation stage.
After 4 hours, the wells were topped up with DMEM containing Turbofect transfection reagent.
The average value of the three wells for each experimental condition was measured and recorded.
In some cases, the above experiment was undertaken using a first type of pulsed technology where only a single transmitter was provided in the pulsed device (Technique 1 pulsed technology). In some cases, the above experiment was undertaken using a second type of pulsed technology where an array of multiple transmitters was used in the pulsed device (Technique 2 pulsed technology). In particular, in experiments using the Type 2 pulsed technology, six transmitters were provided and each transmitter was arranged centrally or substantially centrally of four wells of a 24 well plate when the plate was located on the pulsed device.

Luciferase Assay Protocol—Using the Dual-Luciferase Reporter Assay System (Promega, USA)

The following link sets out the protocol used but a summary of the protocol is set out below. https://www.promega.co.uk/products/luciferase-assays/reporter-assays/dual_luciferase-reporter-assay-system/?catNum=E1910#protocols

Method Steps
1. 24 hours after the transfection experiments took place, the media was removed from the cells.
2. The cells were washed twice with Phosphate Buffered Saline (PBS).
3. 100 μL of 1× Passive Lysis Buffer (Promega, USA) was added to the cells.
4. The cells were incubated for 15 minutes at 37° C. while gently rocking on a Belly Dancer® Orbital Shaker (Sigma, Aldrich).
5. 10 μL of cells was taken from each well and placed in a white 96 well plate.
6. The cells were analysed with a Microplate Luminometer LB 96V (EG & G Berthold, Germany) using the Dual-Luciferase Assay System Protocol (Promega, USA).
7. The analysis was undertaken by injecting 30 μL Luciferase Assay Reagent II (Promega, USA) to measure firefly luciferase activity and then 304, Stop & Glo™ reagent to block firefly luciferase and measure Renilla luciferase activity.
8. Lysed extracts were then kept at −20° C. to run Western Blots if required.
9. Transfection Efficiency in cells was undertaken by placing the cells in an Incucyte® Live Cell Analysis System (Sartorius, Germany) for 72-96 hours and their fluorescence is measured every hour. Data was collected and analysed in Excel®.

### Results for Experiment 1

Table 1 shows the results of the CHO K1 cell experiments where technique 1 pulsed technology was used with the Turbofect amphiphilic construct and associated methodology.

TABLE 1
(Technique 1 Pulsed Technology)

| Technique 1 & | | Pulsed |
|---|---|---|
| Turbofect | Control | Technology |
| Luminescence | 40147 | 131502 |
| (a.u.) | | |
| Luminescence | 62199 | 100925 |
| (a.u.) | | |
| Luminescence | 94460 | 117862 |
| (a.u.) | | |
| Average | 65602 | 116763 |
| Luminescence | | |
| (a.u.) | | |

% Increase in Pulsed Technology compared to Control - 178%
Average Fold Increase in Pulsed Technology compared to Control - 1.8
T-test - 0.024

Table 2 shows the results of the CHO K1 cells experiments where technique 2 pulsed technology was used with the Turbofect amphiphilic construct and associated methodology.

TABLE 2
(Technique 2 Pulsed Technology)

| Technique 1 & | | Pulsed |
|---|---|---|
| Turbofect | Control | Technology |
| Luminescence | 58615 | 94228 |
| (a.u.) | | |
| Luminescence | 73946 | 184908 |
| (a.u.) | | |
| Luminescence | 91469 | 242183 |
| (a.u.) | | |
| Average | 74676.67 | 173773 |
| Luminescence | | |
| (a.u.) | | |

% Increase in Pulsed Technology compared to Control - 232.7%
Average Fold Increase in Pulsed Technology compared to Control - 2.3
T-test - 0.044

Table 3 shows the results of the HCT 116 cells experiments where pulsed technology was used with the Turbofect amphiphilic construct and associated methodology.

TABLE 3

| Technique 1 & | | Pulsed |
|---|---|---|
| Turbofect | Control | Technology |
| Luminescence | 16794 | 23706 |
| (a.u.) | | |
| Luminescence | 14626 | 24841 |
| (a.u.) | | |
| Luminescence | 15555 | 16510 |
| (a.u.) | | |
| Average | 15658.33 | 21685.67 |
| Luminescence | | |
| (a.u.) | | |

% Increase in Pulsed Technology compared to Control - 138.5%
Average Fold Increase in Pulsed Technology compared to Control - 1.4
T-test - 0.044

With reference to Tables 1 and 2 and FIGS. 4a and 4b, the transfection efficiency in CHO K1 Cells associated with the Turbofect amphiphilic construct are shown for controls and pulsed technologies according to the present invention (Pulzar). Each conditions contains three replicates. The amount of luminescence was measured for all cells as a measure of luciferase activity (i.e. transfection).

It can be seen that the transfection efficiency in CHO K1 cells using technique 1 pulsed technology was significantly improved compared to the control cells, with a t-test value of 0.024, an average fold increase of 1.8 and % increase of 178.0.

It can also be seen that the transfection efficiency in CHO K1 cells using technique 2 pulsed technology was significantly improved compared to the control cells, with a t-test value of 0.044, an average fold increase of 2.3 and % increase of 232.7.

Furthermore, it can be seen that experiments undertaken with technique 2 pulsed technology (i.e. the 6 electronic transmitter chip array) produced significantly better results than the experiments undertaken using technique 1 pulsed technology.

With reference to Table 3 and FIG. 4c, the transfection efficiency in HCT 116 Cells associated with the Turbofect amphiphilic construct are shown for controls and pulsed technology according to the present invention (Pulzar). Each condition contains three replicates. The amount of luminescence was measured for all cells as a measure of luciferase activity.

It can be seen that the transfection efficiency in HCT 116 cells using pulsed technology was significantly improved compared to the control cells, with a t-test value of 0.044, a fold increase of 1.4 and % increase of 138.5.

Thus, it can be concluded that the pulsed technology of the present invention significantly increased the transfection efficiency in adherent CHO K1 cells and HCT 116 cells compared to when pulsed technology was not used. Furthermore, six electronic transmitters produced a further increase in transfection efficiency compared to where only a single electronic transmitter was used.

### Experiment 2—Transfection of Adherent HCT Cells Using Either the IGFBP3 Promoter Containing Plasmid or the SV40 Promoter Containing Plasmid, and PEI as the Amphiphilic Construct

Experiment 2 was undertaken to look at the effect of the pulsed technology of the present invention on the process of transfection of adherent HCT116 (Human Colon Cancer Cell Line) (ATCC, USA) using the Adluc and Renilla Plasmids containing either the IGFBP3 promoter or the SV40 promoter in PEI (Fisher Scientific, USA) amphiphilic constructs. The methodology of Experiment 1 was followed for Experiment 2.

### Results for Experiment 2

Table 4 shows the results of the HCT 116 cells experiments for the IGFBP3 promoter using the PEI amphiphilic construct and associated methodology.

TABLE 4

| Average | | |
|---|---|---|
| Luceiferase | | Pulsed |
| activity | Control | Technology |
| Exp 1 | 1179 | 1753 |
| (Replicate) | 827 | 1918 |
| | 1124 | 1865 |
| Exp 2 | 1190 | 1732 |
| (Replicate) | 1831 | 2857 |
| | 1325 | 2472 |
| Average | 1246 | 1099.5 |
| St. Dev | 330.616394 | 459.4809028 |

% Fold Increase = 168.4991974
t.test p < 0.004154274

TABLE 5

| Average | | |
|---|---|---|
| Luceiferase | | Pulsed |
| activity | Control | Technology |
| Exp 1 | 9204 | 11682 |
| (Replicate) | 6769 | 12714 |
| | 5370 | 13356 |
| Exp 2 | 6291 | 9838 |
| (Replicate) | 15530 | 14261 |
| | 7637 | 17011 |
| Average | 8466.833 | 13143.66667 |
| St. Dev | 3696.691138 | 2428.921626 |

% Fold Increase = 155.2371016
t.test p < 0.026953884

Table 5 shows the results of the HCT 116 cells experiments for the SV40 promoter using the PEI amphiphilic construct and associated methodology.

With reference to Tables 4 and 5 and FIGS. 5a and 5b, the transfection efficiency of DNA plasmids, containing either the IGFBP3 promoter or SV40 promoter, and associated with a PEI amphiphilic construct, in HCT 116 Cells are shown for controls and pulsed technologies according to the present invention (Pulzar). Each graph contains two experiments which are replicates of three. The amount of luminescence was measured for all cells as a measure of luciferase activity.

It can be seen that the transfection efficiency (shown by the IGFBP3 promoter) in HCT 116 cells using pulsed technology was significantly improved compared to the control cells, with a t-test value of 0.004 and % increase of 168.5.

It can be seen that the transfection efficiency (shown by the SV40 promoter) in HCT 116 cells using pulsed technology was significantly improved compared to the control cells, with a t-test value of 0.027 and % increase of 155.2.

Thus, it can be concluded that the pulsed technology of the present invention significantly increased the transfection efficiency in adherent HCT 116 cells compared to when pulsed technology was not used.

### Experiment 3—Transfection of Suspension HEK 293Freestyle Cells Using the GFP Plasmid and PEI as the Amphiphilic Construct

This experiment was undertaken to look at the effect of the pulsed technology of the present invention on the process of transfection of Human Embryonic Kidney (HEK) suspension cells 293Freestyle using a green fluorescent protein (GFP) plasmid in a PEI amphiphilic construct. The pulsed technology was applied to the cells and the transfection reagent during the transfection process only.

Consumables

Opti-MEM™ I Reduced Serum Media (Thermo Fisher, USA)

Green Fluorescent Protein (GFP) plasmid (made by Dundee University, UK)

293-Freestyle Suspension Cells (Thermo Fisher, USA)

293-Free Expression Media (Sigma-Aldrich, USA)

Alfa Aesar™ Polyethyleneimine, linear, M.W. 25.00 (PEI) (Fisher Scientific, USA)

Method Steps when Pulsed Technology Used on Reagent and Cell Mixture Only
1. Seed 6×10⁵-7×10⁵ 293-F cells/mL the day before transfection.
2. Count the number of cells on the day of transfection and dilute cells if necessary to have a density of 1×10⁶ cells/mL
3. Transfect 15 μg of the Green Fluorescent Protein (GFP) plasmid/flask with 30 μL of 293-Free Expression Media/flask
4. Use a Ratio of DNA:PEI of 1:2.
5. Use 293-Free Expression Media following the manufacturers instructions (https://www.sigmaaldrich.com/content/dam/sigma-aldrich/docs/SAJ/Brochure/5TB88483.pdf)—User Protocol TB515 Rev. B 0411JN [4]
6. In order to prepare the DNA-Transfection Mixture:
   Add 2.4 mL of Opti-MEM into a flask
   Add 30 μg of GFP plasmid to the flask
   Add 60 μL of 293-Free Expression Media
   Divide the resulting mixture volume into two 125 mL Erlenmeyer flasks, each containing 1×10⁶ cells/mL in 28.8 mL of 293 Expression Media;
   Incubate the two flasks at 37° C. at 8% CO² on a Bellydancer Orbital Shaker (Sigma, Aldrich) at 125 rpm in two separate incubators. Pulse one of the flasks for 3 hours using the Pulsed Technology according to the present invention in one of the incubators and incubate the other flask without any Pulsed Technology in the second incubator. After 3 hours, place both flasks in the same incubator without any Pulsed Technology to allow transfection efficiency to be measured over time for as long as required (120 hours in the case of the experiment).

### Experiment 3 Results

FIG. 6 shows the maximum improvement of transfection efficiency achieved in the experiments using the pulsed technology methodology set out herein.

FIG. 7 shows the average improvement of transfection efficiency using the pulsed technology of the present invention.

With reference to FIGS. 6 and 7, the transfection efficiency of a GFP plasmid associated with a PEI amphiphilic construct in HEK 293 Freestyle Suspension Cells are shown for controls and pulsed technologies according to the present invention (Pulzar). The Pulzar and no Pulzar (control) exposure was performed prior to the growth curves visualised in the graphs of FIGS. 6 and 7.

It can be seen that the transfection efficiency (shown by the amount of the mean Green Fluorescence measured) in HEK 293 Freestyle Suspension Cells using pulsed technology was significantly improved compared to the control cells, with a t-test value of less than 0.05 and a peak increase of 2.3 fold more GFP expression was observed, as shown in FIG. 6.

It can be seen that the transfection efficiency (shown by amount of the mean Green Fluorescence measured) in HEK 293 Freestyle Suspension Cells using pulsed technology was significantly improved compared to the control cells, with a t-test value of less than 0.05 and an over 50% increase in GFP expression was observed, as shown in FIG. 7. The delta was calculated to mark the % increase in GFP expression throughout the time period of the experiment.

Thus, it can be concluded that the pulsed technology of the present invention significantly increased the transfection efficiency in HEK 293 Freestyle Suspension Cells compared to when pulsed technology was not used.

### Experiment 4—Transfection of Suspension Jurkat E6 Cells Using Adluc and Renilla Plasmids and Using Either PEI or TransIT2020 as the Amphiphilic Construct

This experiment was undertaken to look at the effect of the pulsed technology of the present invention on the process of transfection in Jurkat E6 Cells (Human leukaemic T-Cell lymphoblast cells) (European Collection of Authenticated Cell Cultures (ECACC), UK) using Adluc and Renilla Plasmids in either PEI (Fisher Scientific, USA) or TransIT2020 (Mirus Bio, USA) amphiphilic constructs. The pulsed technology was applied to a) the cells and the transfection mixture (the transfection complex) during the transfection process only; and b) the transfection mixture prior to forming a transfection complex with the cells and then to the transfection complex during the transfection process.

Consumables

Opti-MEM™ I Reduced Serum Media (Thermo Fisher, USA)

Fetal Calf Serum (FCS) (Hyclone, USA)

RPMI Medium (Sigma-Aldrich, UK)

2×24 Well Plates Nunc (1.9 cm²/well) (Thermo Fisher, USA)

1 μg of AdLuc plasmid/well (Luciferase expressing plasmid/DNA) (made by Dundee University, UK)

80 ng Renilla plasmid/well (Luciferase expressing plasmid/DNA) (made by Dundee University, UK)

Alfa Aesar™ Polyethyleneimine, linear, M.W. 25.00 (PEI) (Fisher Scientific, USA)

TransIT2020 (Mirus Bio, USA)

Method Steps

Control—Using PEI

1. 650 μL of Opti-MEM media was mixed with 13 μg of AdLuc plasmid and 1 μg of Renilla plasmid in a first tube;

2. 650 μL of Opti-MEM media was mixed with 42 μg of PEI in a second tube;

3. The contents of the second tube was mixed in a dropwise manner to the first tube while gently vortexing until a final volume of 1.3 mL mixture was achieved using a Vortex-Genie 2, Model G560E, (Scientific Industries, USA);

4. The transfection mixture was incubated for 15 minutes at room temperature (approx. 20° C.);

5. 100 μL of this incubated transfection mixture was then dispensed into wells labelled A1-A6 on each of the two 24 well plates (Plates 1 and 2). This formed the transfection mixture.

Invention—with Pulsed Technology Using PEI on Transfection Mixture Prior to Transfection Complex being Created

1. Then, steps 1-3 above were repeated but at step 4—the mixture forming the transfection mixture was incubated for 15 minutes at room temperature (approximately 20° C.) by locating the first tube on a pulsed electromagnetic signal device according to the present invention. The pulsed device operates as described above (i.e. pulsed device operated at 2.45 GHz+/−50 MHz, at power 2 mW using a pulsed frequency of 15 Hz).

2. 100 μL of this incubated pulsed transfection mixture was dispensed into wells labelled B1-B6 on each of the two 24 well plates (Plates 1 and 2);

Control Using TransIT2020
5. 700 μL of Opti-MEM media was mixed with 13 μg of AdLuc plasmid and 1 μg of Renilla plasmid in a first tube;
6. 42 μL of TransIT2020 was added and mixed by vortexing using a Vortex-Genie 2, Model G560E, (Scientific Industries, USA);
7. The transfection mixture was incubated for 15 minutes at room temperature (approximately 20° C.)
8. 50 μL of this incubated transfection mixture was dispensed into wells labelled C1-C6 on each of the two 24 well plates (Plates 1 and 2);
   Invention—Using TransIT2020 with Pulsed Technology on Transfection Mixture Prior to the Transfection Complex being Created
3. Steps 1-2 above were repeated for the TransIT2020 Control. At step 3 the transfection mixture was incubated for 15 minutes at room temperature (approx. 20° C.) by locating the first tube on a pulsed electromagnetic signal device according to the present invention. The pulsed device operated at 2.45 GHz+/−50 MHz, at power 2 mW using a pulsed frequency of 15 Hz.
4. 50 μL of this incubated pulsed transfection mixture was dispensed into wells labelled D1-D6 on each of the two 24 well plates (Plates 1 and 2);

Cell Lines Added to Plates 1 and 2
For the Plates 1 and 2, a transfection complex was created by adding the Jurkat E6 cells in RPMI and 10% FCS into each well of the two 24 well plates at 2×10⁵ cells/well and then made up to a final volume of 600 μL.
Plates 1 and 2 were incubated in an incubator at 37° C., 5% CO² overnight;
In plate 1 there was no pulsed technology given to the transfection complex during the overnight incubation stage, whereas plate 2 was subjected to pulsed technology according to the present invention for 3 hours during the overnight incubation stage.
The average value of the three wells for each experimental condition was measured and recorded.

Luciferase Assay Protocol—Using the Dual-Luciferase Reporter Assay System (Promega, USA)

Method Steps—as Set Out Above

### Results for Experiment 4

TABLE 6

| Average | | |
|---|---|---|
| Luceiferase | | Pulsed |
| activity | Control | Technology |
| Luminescence | 15840.33 | 26452.00 |
| (a.u.) Exp A | | |
| Luminescence | 15840.33 | 31919.00 |
| (a.u.) Exp B | | |
| Luminescence | 15840.33 | 35771.67 |
| (a.u.) Exp C | | |

Table 6 shows the results of the Jurkat E6 cells experiments for the AdLuc and Renilla Plasmids using the PEI or TransIT2020 amphiphilic constructs and associated methodology.

Exp A— where pulsed technology was applied to the transfection complex only (i.e. once the transfection mixture had been added to the cells and during incubation).

Exp B—where pulsed technology was applied to the transfection mixture (prior to adding the Jurkat E6 Cells) only.

Exp C—where pulsed technology was applied to the transfection mixture prior to adding the Jurkat E6 Cells) and then also to the transfection complex (i.e. once the transfection mixture had been added to the cells and during incubation).

### Results for Experiment 4

With reference to Table 6 and FIG. 8, each bar on the graph represents an average of 3 replicates. A 1.7 fold increase in transfection efficiency was observed when the transfection complex only received the pulsed technology. A 2.0 fold increase in transfection efficiency was observed when the transfection mixture only received the pulsed technology. A 2.3 fold increase in transfection efficiency was observed when both the transfection mixture and the transfection complex received the pulsed technology. Therefore, it can be concluded that the use of the pulsed technology according to the present invention significant increased transfection efficiency both when used on the transfection mixture or transfection complex alone, but further increases in transfection efficiency were observed when the pulsed technology was applied to both the transfection mixture and the transfection complex.

In one embodiment of the present invention, as shown in FIGS. 9a and 9b, there is provided apparatus 301 in the form of an electronic device that can be used for improving transfection efficiency and/or intra-cellular delivery of one or more agents for providing one or more therapeutic methods of treatment to a patient, for increasing delivery of a pharmaceutical and/or therapeutic agent into a patient, for increasing and/or decreasing gene expression, protein expression and/or the like.

The apparatus 301 is capable of emitting pulsed electromagnetic signals at a pre-determined frequency, at a pre-determined pulse rate, at a pre-determined power level and for a pre-determined period of time as previously described. However, this apparatus 301 can be worn adjacent a patients body to allow the pulsed electromagnetic signals to be directed towards the patients body in use. The pre-determined parameters can be pre-set by the manufacturer or can be user selectable as required.

The apparatus 301 includes a housing 302, which includes a pulsed signal transmission system. In particular, in this example, the pulsed signal transmission system includes a circuit board 307 with transmission means in the form of an electronic transmission chip 304, typically provided as part of an integrated circuit, which allows the transmission of pulsed electromagnetic signals when the device is operational in use.

In one example, the housing includes a base surface 303, an upper surface 311 opposite to base surface, and one or more side walls 313 located between the upper and base surfaces 311, 303 respectively.

Control means in the form of a control unit 310 can be provided to allow the selective operation of the apparatus 301. A memory device 306 is provided to allow data, one or more operating parameters, software and/or the like to be stored and retrieved when necessary. The control unit preferably includes micro-processing means to allow processing of data and/or the like.

The apparatus 301 could also include one or more power cells 310 to provide electrical power to the apparatus. A rechargeable facility can also optionally be provided to allow the power cells to be recharged from a remote power source rather than having to be replaced.

The electronic transmission chip 304 is arranged in the housing 302 to emit the pulsed electromagnetic signals from the apparatus 301 in a particular direction or directions use. The direction of transmission of the pulsed electromagnetic signals will typically depend on what purpose the apparatus 1 is being used for. If the apparatus is being used for wearing by a user, the signals are typically directed through base surface 303 towards the user.

In one embodiment of the present invention, the electronic transmission chip is arranged in the housing 302 such that it is spaced less than 5 cm from the surface of the housing 302 that is to be brought into contact with a users skin in use, and preferably approximately 1 cm. This allows the electromagnetic signals emitted from the chip to be directed to the patient in use.

The apparatus of the present invention is designed to be used at room temperature (i.e. approximately 20° C.), in temperatures colder than room temperature and/or can be used at temperatures above room temperature, such as for example in a patients body.

In one embodiment, the control unit 310 is programmed to control the transmission chip to allow it to emit pulsed electromagnetic signals at a frequency of 2.45 GHz+/−50 MHz, at a pulsed frequency of 15 Hz and at a power of approximately 2 mW. It will be appreciated that the parameters associated with the pulsed electromagnetic signals can be adjusted and/or be user selectable as required. For example, the time for which the pulsed electromagnetic signals are emitted can be selected by the user if required. In addition, the power can be adjusted, although it typically remains in the milliwatt range so as to avoid over energising the cells contained within the container 16 in use. In one example, the pulsed signals last for 1 ms and the rest period between signals is 66 ms. This provides a duty cycle of less than 2%.

However, it should be noted that any frequency transmission in the Industrial, Medical and Scientific frequency bands (i.e. 2.4 to 2.4835 GHz, preferably 2.45 GHz+/−50 MHz) could be possible by the electronic apparatus in use.

In one example, the electromagnetic signals are RF signals using the Bluetooth LE protocols advertising feature and are transmitted using GFSK between 0.45 and 0.55. However, it should be noted that any frequency transmission in the Industrial, Medical and Scientific frequency bands could be possible by the electronic apparatus in use.

In the illustrated example in FIG. 9a, selection means 305 are provided to allow the selection of a particular sequence of pulses, frequency, timing, and/or strength of the pulses in order to allow the apparatus to be configured according to a users requirements.

In the embodiment shown in FIGS. 9a and 9b, the apparatus 301 is illustrated for positioning directly on the surface of a patients skin 312. In this example, attachment means in the form of a band 314 is provided for detachably attaching the apparatus 301 to the users body. More particular, band 314 passes around the patients arm or limb so as to secure the housing 302 in the required location with respect to a portion of the patients skin. Alternatively, the base surface 303 of the housing which is to contact with the skin can be provided with an adhesive material thereon to allow the same to be adhered to the patients skin at the required location. When the apparatus 301 is operated in use, the pulsed electromagnetic signals 322 emitted from the housing 302 pass into at least a portion of the patients skin, and possibly further into the tissue 324 and cells of the patients body.

In another embodiment of the present invention, as shown in FIGS. 10a and 10b, the apparatus housing 302 is located on top of a drug-delivery “patch”325 (sometimes referred to as a ‘transdermal patch’) which, in turn, is adhered to a portion of a users skin 312. In this embodiment the pulsed electromagnetic signals 322 are emitted from the housing 302, are directed into the patch 325 and through the portion of the patch which includes the agent or drug 326 to the skin 312. The drug is delivered into the users tissue and cells 324 by passing through the users skin. Use of the pulsed electromagnetic signals enhances the absorption and uptake of the drug through the users skin. Reference to “drug” can mean any agent, pharmaceutical and/or therapeutic agent as required.

In another embodiment of the present invention, as shown in FIG. 11, the apparatus is provided as an implantable device. More particularly, the housing 302 of the apparatus provides a sterile outer casing which is implanted subcutaneously under the users skin 312 and/or in the users tissue 324. Once implanted, the apparatus emits the pulsed electromagnetic signals 322 therefrom. The implant is positioned so that the signals 322 are emitted in a desired direction towards, for example, a cancerous tumour 328.

In yet further embodiment of the present invention, as shown in FIGS. 12a and 12b, the apparatus is provided in the form of a pendant 336. In the illustration, the pendant is arranged to be worn on a chain 337 so as to position the pendant the level of the throat/upper chest 338 of the patient or person 339. The pulsed electromagnetic signals 322 are then directed from the pendant into the body of the wearer as indicated by arrow 341 of FIG. 12a. The face 343 of the pendant 336 is arranged to be locatable closest to the person when the pendant is worn at the required location.

In one example, the apparatus of the present invention could be worn so as to minimise viral replication and as a means to provide greater immunological protection to the wearer. Thus, in this embodiment, when the pendant 336 is worn at the level of throat/upper chest, a boost is provided to the immunity of this critical respiratory zone in the wearer.

Typically, in whichever embodiment, the apparatus of the present invention is provided at or adjacent a portion of the skin of a user which has been selected to provide a topical and focused treatment at a predetermined location.

For example, if the purpose of the apparatus is to provide a treatment for a cancerous tumour in a patient, the apparatus is located in the vicinity of, or is implanted into, a recognised cancerous tumour such as may be present, for example, in the liver, kidney, breast or bone. Alternatively, if the apparatus is to be provided to achieve a therapeutic benefit or to limit or prevent the possibility of infection, the apparatus can be located externally of the patient adjacent the portion of the patients body at which therapeutic or preventative effect is believed to be most beneficial, such as at the throat region of the patient or person.

Thus, if the apparatus is located directly on the skin 312 of a patient, the pulsed electromagnetic signals are emitted through the skin and into the tumour to provide a change in condition of the tumour cells. If the apparatus is to be used in conjunction with a patch or other drug carrying item, such as for example as shown in FIGS. 10a and 10b, then the drug is enabled to pass through the patients skin more easily than would conventionally be possible. The pulsed electromagnetic signals are thought to increase the size of the skin pores and allow greater space for the passage of the drug therethrough. Thus, pharmaceutical drugs or other agents can be delivered more efficiently and effectively using the present invention. In addition, pharmaceutical drugs or other agents which cannot currently be provided transdermally, can now be supplied into the body using the process of the present invention. The provision of the apparatus of the present invention enhances both delivery of the drug by increased skin permeability and provides a direct treatment benefit.

Although the above examples shows transfection of an agent in the form of nucleic acid associated with an amphiphilic construct in different types of eukaryotic cells being significantly improved following exposure to the pulsed technology of the present invention at different stages of the transfection process, the Applicants fully expect and predict that the transfection and/or intra-cellular delivery of one or more pharmaceutical and/or therapeutic agents or compounds, small molecules or small molecular material of less than 5 Kilodaltons, large molecules or large molecular material of greater than or equal to approximately 5 Kilodaltons, one or more proteins, vaccine, an organic agent, and/or one or more antibodies when associated with an amphiphilic construct, to be significantly improved on exposure of the same to the pulsed technology of the present invention in one or more eukaryotic cells. These predictions and expectations are based on data already collected by the Applicants in their co-pending application claiming priority from British Patent Applications GB2004411.1, GB2009297.9, GB20044112.9 and GB2009296.1, the content of which is incorporated herein by reference, which shows that the intra-cellular delivery of a “naked agent” in the form of Doxorubicin (not associated with an amphiphilic construct) in eukaryotic cells is significantly improved when exposed to the pulsed technology of the present invention. The data for these experiments is reproduced below to show support for the breadth of the claim set of the present application. The Applicants predict the same or similar mechanism of improvement of transfection efficiency and/or intra-cellular delivery when an agent is associated with an amphiphilic construct as when a “naked agent” (i.e. not associated with an amphiphilic construct) is used. This is because the pulsed electromagnetic waves or signals according to the present invention are thought to be sufficient to rotate H₂O periodically around its dipole with relatively long rest or relaxation periods. The periodic rotation of H₂O is thought to interrupt hydrogen bonding in the phospholipid bilayer or cell membranes of the eukaryotic cells. This periodic or intermittent low energy perturbation of the cell membranes is thus thought to stimulate increased interaction with the agent, some molecules and/or cell membranes and their environment, such as for example, the nucleic acid or agent with the cell membrane. The relatively long rest or relaxation period between the pulses of the pulsed electromagnetic signals is thought to be sufficient to maintain cellular integrity.

In the following experiments taken from the Applicants co-pending patent application, the same pulsed technology of the present invention was used on a “naked agent” in the form of Doxorubicin when added to a eukaryotic cells line.

Human Colon Tumour (HCT) 116 cells (adherent cells) (ATCC, USA-ATCC® CCL-247™) were seeded at a density of 3×10⁵ cells per well in two CELLSTAR®6-well plates (9.6 cm²) in a final volume of 5 mL Dulbeccos Modified Eagle Medium (DMEM) (Thermo Fisher, USA)+10% Fetal Bovine Serum (FBS) (Hyclone, USA) 24 hours before treatment.

The naked agent used was Doxorubicin (0.25 μM) (Sigma Aldrich) in absolute ethanol and was given to the cells for a 1 hour treatment period and incubated at 37° C., at 5% CO².

After treatment the media was removed and fresh media was added to the cells. One of the plates was incubated directly at 37° C., at 5% CO² and the second plate was placed in a different incubator and pulsed using the pulsed technology of the present invention at 37° C., at 5% CO².

Protein extracts were collected at 3 hours, 6 hours, 9 hours, 16 hours or 24 hours of treatment for analysis by SDS-page.

The following Western Blot protocol is set out in reference [5].

Preparation of Protein Extracts for Western Blot

1. For protein extraction the cells were washed twice with ice-cold PBS and then lysed in NP-40 extraction buffer (50 mM Tris ph 7.5; 10% glycerol; 0.1% “NP-40 Alternative” (Merck Millipore, USA); 100 mM NaCl; 0.2 mM EDTA) supplemented with 1× Complete™ Protease Inhibitor Cocktail (Roche, Switzerland). Extracts were sonicated (20 seconds, 20% amplitude) and protein concentration was determined using BCA™ Protein Assay Kit (ThermoFisher Scientific USA) according to the manufacturers recommendations.

Western Blot Protocol

1. Protein extracts (15/20 μg depending on the experiment) were supplemented with 0.1M dithiothreitol (DTT) and 1×LDS buffer (Invitrogen, USA) and were heated at 95° C. for 10 min before loading on NuPAGE 10% Bis-Tris polyacrylamide gels (Invitrogen, USA).

2. Protein samples were separated by electrophoresis (100V) using 1×MOPS Running Buffer. Transfer of proteins was performed at 12V overnight onto a nitrocellulose membrane (Protran 0.1 μm from GE Healthcare, USA) in 1× Transfer Buffer supplemented with 20% methanol. 1× Transfer Buffer is prepared from 10× Wet blot solution containing 144 g of glycine and 30 g Tris-Base in a final volume of 1 L milli-Q water.

3. Membranes were blocked for 30 min in 5% BSA diluted in PBS—0.1% Tween20 before being incubated overnight with a primary antibody (Mouse monoclonal antibody D01). After a wash of 15 min. in PBS-Tween20, membranes were incubated for 1 h with a corresponding secondary antibody (HRP conjugated Donkey anti Mouse). All secondary antibodies, conjugated with Horse Radish Peroxidase (HRP), were purchased from Jackson ImmunoResearch lab and used at 1:10000/1:15000 dilution (depending on the antibody) in 5% BSA PBS-Tween20.

At the end of the incubation membranes were washed twice with PBS-Tween20 for 15 min followed by a final 10 min. wash with PBS. The chemiluminescence signal was detected on Hyperfilm™ ECL (Cytiva, USA) using the Amersham ECL Western Blotting Detection System (Cytiva, USA).

Results

Referring to FIG. 13, it can be seen from the Western Blot that p53alpha—the main isoform of the p53 protein—was upregulated after treatment with the pulsed technology of the present invention. The effect was observed as soon as 3 hours after the addition of the drug and was most evident 24 hours post-treatment. Other isoforms of p53 were also more upregulated under the effect of the pulsed technology according to the present invention following doxorubicintreatment, namely d133p53alpha, d133p53beta and d160p53beta. In the Western Blot, [symbol] H2AX was used as a marker to ensure that if any effect was observed it was not caused due to ionising radiation. [symbol] H2AX's expression changes when ionising radiation is present, and since there is no observed change between the pulsed technology according to the present invention and the control arms, it was concluded that the pulsed technology of the present invention did not emit ionising radiation.

Ku80 was used as the loading control to ensure that equal concentrations of each sample was loaded onto each well. Equal concentrations of Ku80 make the rest of the bands in the Western Blot comparable.

Referring to FIG. 14, in another experiment, some cells were treated by the pulsed technology of the present invention and some cells received no pulsed technology of the present invention as a control for 5 days without the addition of doxorubicin. No change in p53alpha expression was observed. When 0.25 μM doxorubicin was added to the cells for 1 hour, the cells under the effect of the pulsed technology according to the present invention showed a significant overproduction of p53alpha compared to the control after 16 hours.

In conclusion, there is clear evidence that treating the cells with the pulsed technology according to the present invention increases the ability of the cells to uptake doxorubicin from the media as various p53 isoforms were upregulated more in the pulsed technology arm compared to the control arm. It can be concluded that this effect is not caused by ionising radiation as the radiation marker gH2AX remained unchanged between the pulsed technology arm and the control arm.

Therefore, the combined effect of enhanced delivery of anti-cancer drugs and the direct treatment of pulsed technology according to the present invention affects beneficially the regulation of replication via the p53 oncogene and improves cancer treatment. Moreover, the effect of the pulsed technology of the present invention on non-mutated p53 of healthy cells results in increased repair of these cells.

### REFERENCES

[1]—Gene Therapy—An Industry Coming Of Age—The Cell Culture Dish Inc. 2020 pages 1-49
[2]—Global Manufacturing of CAR T Cell Therapy—Bruce Levine et al; Molecular Therapy: Methods and Clinical Development, Vol. 4, March 207; 92-101; 2017 Novartis Pharmaceuticals Corp.
[3] Efficient Lipid-Mediated Transfection Of DNA Into Primary Rat Hepatocytes—Sheri L. Holmes et al; In Vitro Cell. Dev. Biol. 30; 347-351—May 1995—1995 Society for In Vitro Biology.
[4] Novagen—User Protocol TB515 Rev. B0411JN—pages 1-4—293-Free Transfection Reagent (2011© EM Chemicals Inc).
[5] Bourdon et al., Genes Dev. 2005, PMID 16131611.
[6] Longo P A, Kavran J M, Kim M S, Leahy D J. “Transient Mammalian Cell Transfection With Polyethylenimine (PEI). Methods Enzymol. 2013; 529-227-240. Doi:10.1016/B978-0-12-418687-3.00018-5.

1. A method of improving transfection efficiency in eukaryotic cells, said method including the steps of:
a) providing a transfection mixture including an agent associated with at least one amphiphilic construct suitable for transfection;
b) introducing the transfection mixture to one or more eukaryotic cells to form a transfection complex;
c) allowing the transfection complex to undergo a transfection process to form one or more transfected cells;
characterised in that the method includes the step of directing pulsed electromagnetic signals provided at any or any combination of a pre-determined frequency, at a pre-determined pulse rate, or at a pre-determined power, at the transfection mixture at step a) prior to creating the transfection complex, at the transfection complex in step b), at the transfection complex in step c) and/or at the transfected cell complex after step c).

2. (canceled)

3. The method according to claim 1 wherein the eukaryotic cells are suspended in solution and/or adhered to a substrate and/or are immortal cells, or the cells have been derived from the tissue of a human and/or animal subject, or the eukaryotic cells have been derived from a human and/or animal subject, and comprise T-cells, lymphocytes, granulocytes and/or macrophages.

4. (canceled)

5. The method according to claim 1, wherein the agent in the transfection mixture is any agent suitable for transfection, and/or any or any combination of nucleic acid; deoxyribonucleic acid (DNA); ribonucleic acid (RNA); a combination of DNA and RNA, mRNA, tRNA, siRNA or miRNA, a pharmaceutical and/or therapeutic agent or compound, an agent of therapeutic and/or pharmaceutical interest, a small molecule or small molecular material of less than 5 Kilodaltons, a large molecule or large molecular material of greater than or equal to approximately 5 Kilodaltons, one or more proteins, vaccine, an organic agent, one or more antibodies or in or includes one or more expression vectors.

6. The method according to claim 1 wherein the agent associated with the at least one amphiphilic construct is contained within the amphiphilic construct, it forms a complex with the amphiphilic construct, it is contained on the amphiphilic construct or it is bonded to the amphiphilic construct.

7. (canceled)

8. The method according to claim 1 wherein the amphiphilic construct can include or consist of any or any combination of at least one liposomal material or vehicle, at least one pegylated liposomal material or vehicle, a micelle, a construct having a phospholipid bilayer, a cationic polymer, or polyethylenimine (PEI).

9-10. (canceled)

11. The method according to claim 5 wherein when the agent is nucleic acid, and the transfection process results in transient expression, or wherein when the agent is nucleic acid and the transfection process results in stable expression, the method further comprises the steps of isolating one or more of the eukaryotic cells after the transfection process, testing expression level of one or more peptides encoded by the agent in the one or more isolated eukaryotic cells or progeny thereof, and selecting one or more isolated eukaryotic cells or progeny based upon the expression level.

12. The method according to claim 1 wherein the step of directing pulsed electromagnetic signals takes place for a pre-determined period of time, or wherein the step of directing pulsed electromagnetic signals takes place for a pre-determined period of time and is approximately 15 minutes when the pulsed electromagnetic signals are directed at the transfection reagent; and/or is approximately 1-4 hours when the pulsed electromagnetic signals are directed at the transfection mixture during or after transfection.

13. (canceled)

14. The method according to claim 1 wherein the pulsed electromagnetic signals are generated by one or more electronic devices, wherein the one or more electronic devices include transmission means or one or more electronic transmission chips for generating and/or transmitting the pulsed electromagnetic signals therefrom in use; wherein each electronic device includes a single transmission means or electronic chip, or each electronic device includes a plurality of transmission means or electronic chips, optionally wherein the electronic device includes a plurality of transmission means or electronic transmission chips and each of said transmission means or electronic transmission chips are arranged a spaced distance apart from one another such that said distance apart equals approximately half of the wavelength of the pulsed electromagnetic signals; or wherein the electronic device includes at least one transmission means or electronic transmission chip per 105 to 115 cm² of a surface of a housing of said device, or a surface of one or more items to be placed upon the electronic device in use, or wherein the electronic device includes six transmission means or electronic transmission chips and said chips are arranged a spaced distance apart from each other in the device such that one transmission means or electronic transmission chip is directed at four wells of a twenty four well plate when said plate is positioned in, on or relative to said electronic device in use.

15. The method according to claim 14 wherein the distance between the transmission means and one or more items receiving the pulsed electromagnetic signals in use is approximately 25 cm or less, approximately 20 cm or less, approximately 15 cm or less, approximately 10 cm or less, approximately 5 cm or less, or approximately equal to 1 cm or less.

16. The method according to claim 1 wherein the pre-determined frequency of the pulsed electromagnetic signals is between approximately 2.4 GHz+/−50 MHz, is between approximately 2.2-2.6 GHz, is at approximately 2.4 GHz+/−50 MHz or is at approximately 2.45 GHz+/−50 MHz, wherein the pre-determined pulse rate of the pulsed electromagnetic signals is approximately 50 Hz or less, approximately 25 Hz or less, approximately 15 Hz or less and/or has a duty cycle of less than 2%, and/or wherein each pulse of the pulsed electromagnetic signals lasts for between approximately 1 ms-20 ms or is approximately 1 ms, optionally wherein the rest period between each pulse of the pulsed electromagnetic signals last for approximately 66 ms or less and/or wherein the pre-determined power provided by each transmission means or electronic transmission chip is approximately +2 dBm to +4 dBm, approximately 1 mW, approximately 2 mW or approximately 2.5119 mW; and/or wherein the pulsed electromagnetic signals are transmitted using Gaussian Frequency Shift Keying (GFSK) between 0.45 and 0.55.

17. (canceled)

18. The method of claim 14 wherein the one or more electronic devices include any or any combination of control means for controlling operation and/or one or more parameters of the electronic device and/or transmission means, power supply means for supplying electrical power to the one or more devices in use, one or more circuit boards, memory means for storing data thereon, user selection means for allowing a user to select the operation, one or more conditions and/or the one or more parameters of the device, or display means for displaying one or more settings, or options for settings.

19. The method of claim 18 wherein the one or more conditions or parameters of the devices that can be selected by a user include any or any combination of the signal frequency, the signal strength, signal or transmission power, the time periods of each pulse or rest period between signal pulses, the signal pulse rate of the pulsed electromagnetic signals.

20. Apparatus for providing improved transfection efficiency in eukaryotic cells, said apparatus including a housing, transmission means located in said housing and arranged to transmit pulsed electromagnetic signals provided at any or any combination of a pre-determined frequency, at a pre-determined pulse rate, or a pre-determined power in use, control means for controlling operation of at least the transmission means in use, and power supply means for providing electrical power to the transmission means and/or control means in use.

21. (canceled)

22. The apparatus according to claim 20, wherein the apparatus comprises at least one transmission means or electronic transmission chip per 105 to 115 cm² of a surface of a housing of said device, or of a surface of one or more items to be placed upon the apparatus in use and/or wherein the apparatus includes a plurality of transmission means or electronic transmission chips and said transmission means or electronic transmission chips are arranged a spaced distance apart such that said distance apart equals approximately half of the wavelength of the pulsed electromagnetic signals.

23. (canceled)

24. The apparatus according to claim 20 wherein the pre-determined frequency of the pulsed electromagnetic signals is between approximately 2.2-2.6 GHz, is approximately 2.4 GHz+/−50 MHz or is approximately 2.45 GHz+/−50 MHz, and/or wherein the pre-determined pulse rate of the pulsed electromagnetic signals is approximately 50 Hz or less, approximately 25 Hz or less, approximately 15 Hz or less and/or has a duty cycle of less than 2%, and/or wherein each pulse of the pulsed electromagnetic signals lasts for between approximately 1 ms-20 ms or is approximately 1 ms, and/or wherein a rest period between each pulse of the pulsed electromagnetic signals last for approximately 66 ms or less and/or wherein the pre-determined power provided by each of said transmission means transmitting said pulsed electromagnetic signals is approximately +2 dBm to +4 dBm, approximately 1 mW, approximately 2 mW or approximately 2.5119 mW, and/or wherein the pulsed electromagnetic signals are transmitted using Gaussian Frequency Shift Keying (GFSK) between 0.45 and 0.55.

25-27. (canceled)

28. The apparatus according to claim 20 wherein the housing comprises an outer casing, wherein at least the outer casing of the apparatus is coated and/or formed from a material to allow the apparatus to be implantable into a persons body or below a users skin in use.

29. The apparatus according to claim 20 wherein the apparatus is provided with at least one holding means or reservoir for holding or containing a transfection mixture which is to be transfected into one or more eukaryotic cells or person in use.

30-31. (canceled)
